(19) United States
(12) Patent Application Publication
(10) Pub. No.: US 2006/0100239 A1
(43) Pub. Date: May 11, 2006

(54) Method for producing optically active amines

(76) Inventors: Izuru Nagasaki, Hiratsuka-shi (JP); Takaji Matsumoto, Hiratsuka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/547,419

(22) PCT Filed: Mar. 3, 2004
(86) PCT No.: PCT/JP04/02690

§ 371 Date: Aug. 31, 2005

(30) Foreign Application Priority Data
Mar. 4, 2003 (JP) 2003-057846
Sep. 22, 2003 (JP) 2003-330769

(51) Int. Cl. C07F 7/02; C07D 215/38; A61K 31/4706

(52) U.S. Cl. 514/313; 546/159; 546/14

(57) ABSTRACT

The present invention provides a method for producing optically active amines of formula (9) or (10): which comprises reacting an imine equivalent of formula (6): with an alkene of formula (7) or an alkyne of formula (8): in the presence of a chiral catalyst, which method does not require additional procedures such as introduction and removal of protecting groups and gives said amines with high purity and high operability. The optically active amines are useful as synthetic 15 intermediates for pharmaceuticals, agrochemicals, etc.

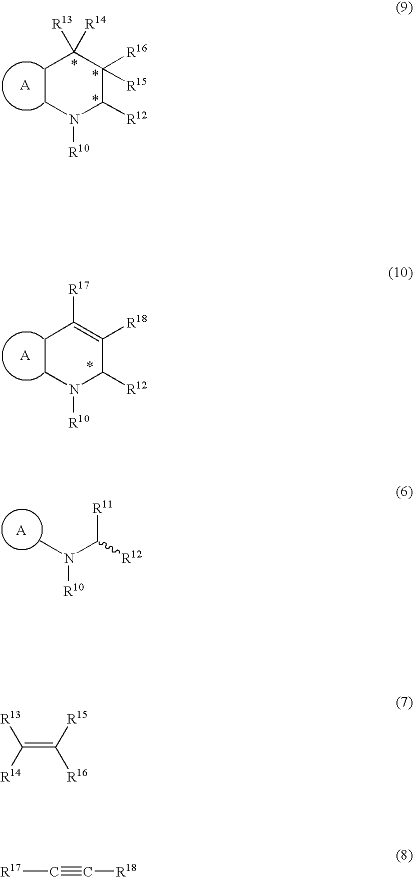

### TECHNICAL FIELD

The present invention relates to a novel method for producing optically active amines, especially dihydroquinolines and tetrahydroquinolines useful, for example, as intermediates for the production of medicines, agricultural chemicals or the like.

### BACKGROUND ART

Recently, amines, especially dihydroquinolines and tetrahydroquinolines are widely used for producing medicines, agricultural chemicals or the like. Up to now, various methods for producing such amines, especially dihydroquinolines and tetrahydroquinolines have been studied.

The methods for producing amines via intermediate amino acid derivatives are disclosed in patent documents 1 and 2, etc. These patent documents disclose the methods for producing amines in which secondary amines obtained by the reaction of primary amines and aryl halides are used. However, in case of introduction of an alkyl or aryl group at the position of the amino group in optically active primary amines such as amino acids or the like, it is required that the reaction conditions which do not cause racemization should be set up. Non-patent document 1 discloses the methods for producing optically active amino acids, wherein the amino group is secondary one by asymmetric nucleophilic addition reaction to imines. However, inflammable diethyl zinc has to be excessively used to obtain the desired amines. This may cause problems in the operation.

Non-patent document 2 discloses the method for producing β-amino acids by asymmetric hydrogenation of enamines, wherein the amino group is protected by acetyl group. However, in the methods mentioned above, it is necessary to protect the secondary amino group with a protecting group such as acetyl group for the asymmetric hydrogenation of enamines. This may cause problems requiring two steps of introduction and removal of such protecting groups. In order to solve such problems, various methods have been studied as disclosed in patent documents 3, 4 and 5.

Patents documents 3 and 4 disclose the method for producing 1,2,3,4-tetrahydroquinoline obtained by reacting an imine equivalent which is obtained by the reaction of amine, aldehyde and benzotriazole, with N-vinyl carbamate in the presence of p-toluenesulfonic acid. Further, patent document 5 discloses the method for producing 1,2,3,4-tetrahydroquinoline obtained by reacting amine with aldehyde and then with N-vinyl carbamate in the presence of boron trifluoride etherate. Furthermore, non-patent document 4 discloses the method for producing 1,2,3,4-tetrahydroquinoline obtained by using chiral Lewis acid. However, these methods could not give quinolines in high optical purity, and thus the methods for producing 1,2,3,4-tetrahydroquinoline in higher optical purity have been desired.

Although each optical purity of cis- and trans-forms of 1,2,3,4-tetrahydroquinoline is described in non-patent document 4, there were drawbacks in that the 1,2,3,4-tetrahydroquinoline obtained by the method described in non-patent document 4 are the mixture of cis- and trans-forms.

The method for producing 1,2,3,4-tetrahydroquinoline by reacting N-benzylidene-2-hydroxyaniline with alkyl vinyl ether in the presence of chiral lanthanide catalyst is described in patent document 6 and non-patent document 8. However, there was a problem that the method described in the non-patent document 8 requires 2-hydroxy group on the aniline to achieve high optical purity.

Non-patent documents 9 and 10 discose the methods for producing N,N-dimethyl-3-(carbazol-9-yl)-3-(benzotriazol-N-yl)propaneamine by reacting an imine equivalent with a vinyl compound such as, for example, 9-vinylcarbazole or the like.

However, there is no description about asymmetric synthesis in the non-patent documents 9 and 10.

Other non-patent documents 3 and 5 to 7 are mentioned below for reference.

Patent document 1: WO02/088069

Patent document 2: WO02/088085

Patent document 3: WO01/40190,

Patent document 4: WO02/13797

Patent document 5: WO00/17164.

Patent document 6: Japanese patent (unexamined) 87628/1998

Non-patent document 1: Chemistry Letters 254-255 (2001)

Non-patent document 2: Tetrahedron Asymmetry, Vol. 2, No. 7. 543-554(1991)

Non-patent document 3: J. Org. Chem., 65, 5009-5013 (2000)

Non-patent document 4: Org. Lett., 1973-1976 (2001)

Non-patent document 5: Tetrahedron Lett., 5765-5768 (1989),

Non-patent document 6: Angew. Chem. Int. Ed., 38, No. 19, 2873 (1999)

Non-patent document 7: J. Am. Chem. Soc., 116, 10520-10524 (1994)

Non-patent document 8: Tetrahedron Lett., Vol. 37, No. 41, 7357-7360(1996)

Non-patent document 9: J. Org. Chem., 58, 812-813, (1993)

Non-patent document 10: J. Org. Chem., 60, 2588-2596, (1995)

### DISCLOSURE OF THE INVENTION

The present invention has been completed on the basis of studies to solve the problems stated above. The object of the present invention is to provide a method for producing the desired optically active amines, especially tetrahydroquinolines and dihydroquinolines, which method does not require additional procedures such as introduction and removal of protecting groups, and gives said amines in high optical purity and high operability.

The present inventors have made intensive studies on the methods for producing optically active amines, especially optically active tetrahydroquinolines and dihydroquinolines. As the result, they have found that the problems as stated above can be solved by reacting a specific imine equivalent or an imine with a specific alkene or alkyne, especially N-vinyl carbamates in the presence of chiral catalyst, especially chiral Lewis acid. The present invention has been completed on the basis of these findings.

Namely, the present invention is shown below.

1) A method for producing optically active amines of formula (9): 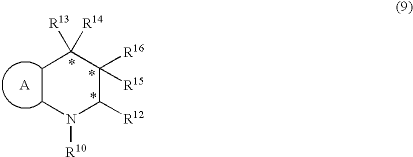
(wherein the group represented by ring A is aryl, substituted aryl, aromatic heterocyclic group or substituted aromatic heterocyclic group; R¹⁰ is hydrogen, alkyl, substituted alkyl, aryl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹² is hydrocarbon, substituted hydrocarbon, COOR¹⁹ (R¹⁹ is a hydrocarbon group or a substituted hydrocarbon group), COR²⁰ (R²⁰ is a substituted amino group) or substituted amino; R¹³, R¹⁵ and R¹⁶ are each independently hydrogen or alkyl; R¹⁴ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, heteroaryloxy, substituted heteroaryloxy, alkylthio, substituted alkylthio, arylthio, substituted arylthio, aralkylthio, substituted aralkylthio, heteroarylthio, substituted heteroarylthio, substituted amino, substituted silyl, alkylseleno, aralkylseleno, arylseleno or heteroarylseleno; R¹⁴ and R¹⁶ taken together may form a ring; the symbol * is an asymmetric carbon atom; 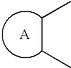
is a divalent group corresponding to the group represented by ring A as mentioned above), or (10): 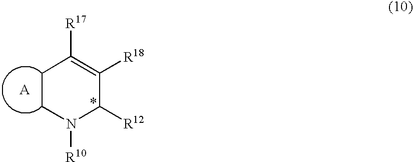
(wherein R¹⁷ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, substituted amino or substituted silyl; R¹⁸ is hydrogen or alkyl; R¹⁰ and R¹² are each the same as mentioned above; the symbol *is an asymmetric carbon atom; and 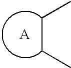
is the same as mentioned above), which comprises reacting an imine equivalent of formula (6): 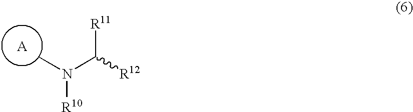
(wherein R¹¹ is a leaving group; and ring A, R¹⁰ and R¹² are each the same meaning as mentioned above) with an alkene of formula (7): 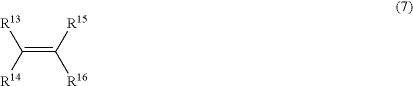
(wherein R¹³, R¹⁴, R¹⁵ and R¹⁶ are each the same as mentioned above) or an alkyne of formula (8):
R¹⁷—C≡C—R¹⁸ (8)
(wherein R¹⁷ and R¹⁸ are each the same as mentioned above) in the 10 presence of a chiral catalyst.

2) The method as described in 1), wherein an optically active compound of formula (9b): 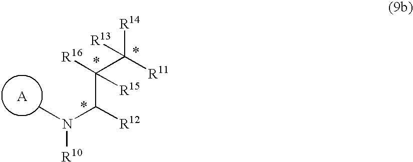
(wherein the symbol * is an asymmetric carbon atom; the group represented by ring A, and R¹⁰ to R¹⁶ are each the same as mentioned above) or an optically active compound of formula (10b): 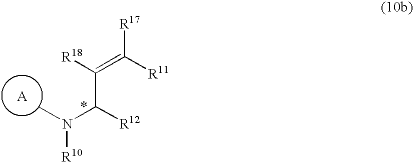
(wherein the symbol * is an asymmetric carbon atom ; the group represented by ring A, R¹⁰, R¹¹, R¹², R¹⁷ and R¹⁸ are each the same as mentioned above) is formed in the reaction system.

3) A method for producing optically active amines of formula (9): 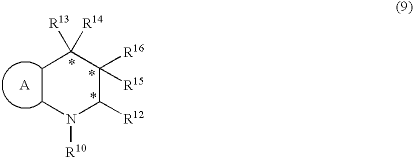
(wherein the group of ring A is aryl, substituted aryl, aromatic heterocyclic group or substituted aromatic heterocyclic group; R¹⁰ is hydrogen, alkyl, substituted alkyl, aryl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹² is hydrocarbon, substituted hydrocarbon, COOR¹⁹ (R¹⁹ is a hydrocarbon group or substituted hydrocarbon group), COR²⁰ (R²⁰ is a substituted amino group), or substituted amino; R¹³, R¹⁵ and R¹⁶ are each independently hydrogen or alkyl; R¹⁴ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, heteroaryloxy, substituted heteroaryloxy, alkylthio, substituted alkylthio, arylthio, substituted arylthio, aralkylthio, substituted aralkylthio, heteroarylthio, substituted heteroarylthio, substituted amino, substituted silyl, alkylseleno, aralkylseleno, arylseleno or heteroarylseleno; and R¹⁴ and R¹⁶ taken together may form a ring; 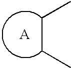
is a divalent group corresponding to the group represented by ring A as mentioned above; and the symbol * is an asymmetric carbon atom) or (10): 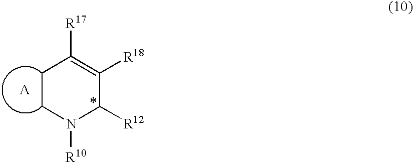
(wherein R¹⁷ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, substituted amino or substituted silyl; R¹⁸ is hydrogen or alkyl; R¹⁰, R¹², ring A, the symbol * and 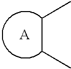
are each the same as mentioned above), which comprises cyclization of an optically active compound of formula (9b): 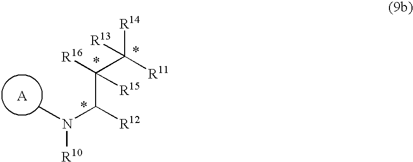
(wherein R¹¹ is a leaving group; the symbol * is an asymmetric carbon atom; the group of ring A and R¹⁰ to R¹⁶ are each the same as mentioned above) or an optically active compound of formula 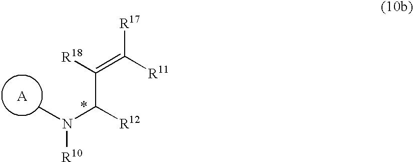
(wherein the group represented by ring A, R¹⁰, R¹¹, R¹², R¹⁷, R¹⁸ and the symbol * are each the same as mentioned above).

4) A method for producing optically active amines of formula (9a-1): 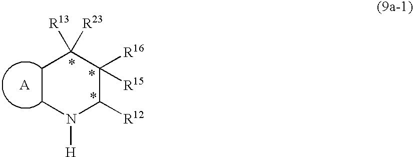
(wherein the group represented by ring A is aryl, substituted aryl, aromatic heterocyclic group or substituted aromatic heterocyclic group; R¹² is hydrocarbon, substituted hydrocarbon, COOR¹⁹ (R¹⁹ is a hydrocarbon group or a substituted hydrocarbon group), COR²⁰ (R²⁰ is a substituted amino group) or substituted amino; R¹³, R¹⁵ and R¹⁶ are each independently hydrogen or alkyl; R²³ is aryl, substituted aryl, aliphatic heterocyclic group, substituted aliphatic heterocyclic group, aromatic heterocyclic group, substituted aromatic heterocyclic group, aryloxy, substituted aryloxy, aralkyloxy, substituted aralkyloxy, heteroaryloxy, substituted heteroaryloxy, alkylthio, substituted alkylthio, arylthio, substituted arylthio, aralkylthio, substituted aralkylthio, heteroarylthio, substituted heteroarylthio, substituted amino, substituted silyl, alkylseleno, aralkylseleno, arylseleno or heteroarylseleno; and R²³ and R¹⁶, taken together may form a ring; 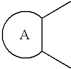
is a divalent group corresponding to the group represented by ring A as mentioned above; and the symbol * is an asymmetric carbon atom) or (10a): 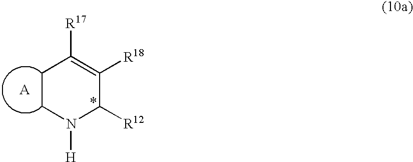
(wherein R¹⁷ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, substituted amino or substituted silyl; R¹⁸ is hydrogen or alkyl; R¹² and 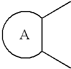
are each the same as mentioned above; and the symbol * is an asymmetric carbon atom), which comprises reacting an imine of formula (6a): 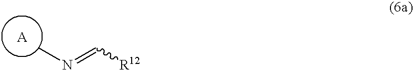
(wherein the group represented by ring A and R¹² are each the same as mentioned above) with an alkene of formula (7a): 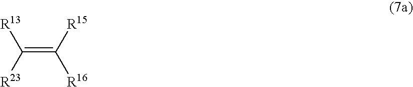
(wherein R¹³, R¹⁵, R¹⁶ and R²³ are each the same as mentioned above), 10 or with an alkyne of formula (8):
R¹⁷—C≡C—R¹⁸ (8)
(wherein R¹⁷ and R¹⁸ are each the same as mentioned above) in the presence of a chiral catalyst.

5) A method for producing optically active 1,2,3,4-tetrahydroquinolines of formula (1A): 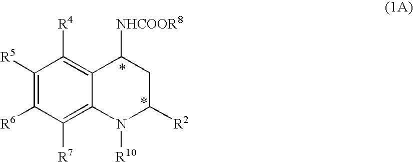
(wherein R² is a hydrocarbon group, a substituted hydrocarbon group or COOR⁹ (R⁹ is a hydrocarbon group); R⁴ to R⁷ are each independently hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R⁸ is a hydrocarbon group; R¹⁰ is hydrogen, alkyl, aryl, substituted alkyl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; and R⁴ and R⁵, R⁵ and R⁶, or R⁶ and R⁷, taken together, may form a fused ring, and the symbol * is an asymmetric carbon atom), which comprises reacting an imine equivalent of formula (2): 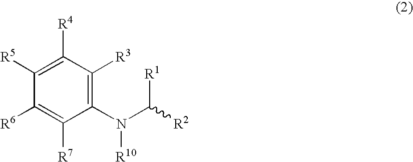
(wherein R¹ is a leaving group; R³ is hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R³ and R⁴, R⁴ and R⁵, R⁵ and R⁶, or R⁶ and R⁷, taken together, may form a fused ring, provided that either of R³ or R⁷ is hydrogen; R² to R⁷ and R¹⁰ are each the same as mentioned above) with a N-vinyl carbamate of formula (3): 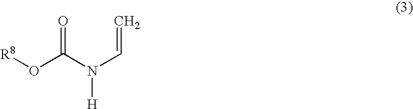
(wherein R⁸ is the same as mentioned above) in the presence of a chiral Lewis acid.

6) A method for producing optically active quinolines of formula (1): 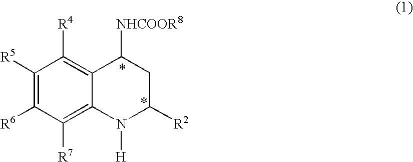
(wherein R² is a hydrocarbon group, a substituted hydrocarbon group or COOR⁹ (R⁹ is hydrocarbon); R⁴ to R⁷ are each independently hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R⁴ and R⁵, R⁵ and R⁶, or R⁶ and R⁷, taken together, may form a fused ring; R⁸ is a hydrocarbon group; and the symbol * is an asymmetric carbon atom), which comprises reacting an imine of formula (2a): 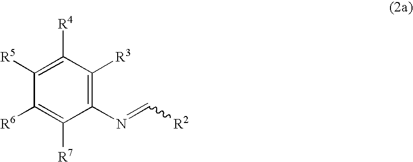
(wherein R³ is hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R³ and R⁴, R⁴ and R⁵, R⁵ and R⁶, or R⁶ and R⁷, taken together, may form a fused ring, provided that either of R³ or R⁷ is hydrogen; R², and R⁴ to R⁷ are each the same as mentioned above) with an N-vinyl carbamate of formula (3): 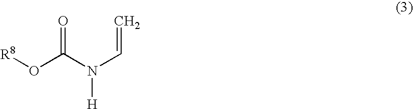
(wherein R⁸ is the same as mentioned above) in the presence of a chiral Lewis acid.

7) A method for producing optically active 1,2,3,4-tetrahydroquinolines of formula (1): 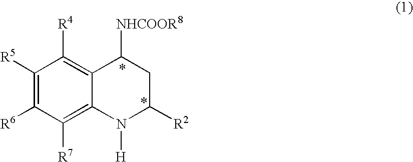
(wherein R² is a hydrocarbon group, a substituted hydrocarbon group or COOR⁹(R⁹ is a hydrocarbon group); R⁴ to R⁷ are each independently hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R⁴ and R⁵, R⁵ and R⁶, or R⁶ and R⁷, taken together, may form a fused ring; R⁸ is a hydrocarbon group; and the symbol * is an asymmetric carbon atom), which comprises reacting an amine of formula (4): 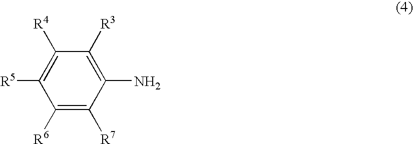
(wherein R³ is hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoly, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R³ and R⁴, R⁴ and R⁵, R⁵ and R⁶, or R⁶ and R⁷, taken together, may form a fused ring, provided that either of R³ or R⁷ is hydrogen; R⁴ to R⁷ are each the same as mentioned above), with an aldehyde of formula (5):
R²—CHO (5)
(wherein R² is the same as mentioned above) and a compound capable of forming imine equivalents, and reacting the resulting imine equivalent of formula (2): 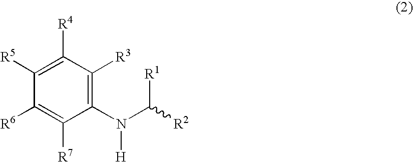
(wherein R¹ is a leaving group; R² to R⁷ are each the same as mentioned above) with an N-vinyl carbamate of formula (3): 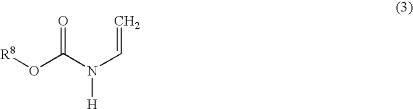
(wherein R⁸ is the same as mentioned above) in the presence of a chiral Lewis acid.

8) A method for producing optically active 1,2,3,4-tetrahydroquinolines of formula (1): 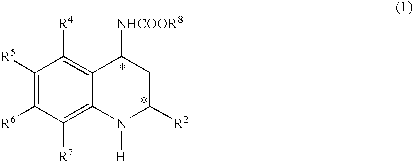
(wherein R² is a hydrocarbon group, a substituted hydrocarbon group or COOR⁹ (R⁹ is a hydrocarbon group); R⁴ to R⁷ are each independently hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R⁴ and R⁵, R⁵ and R⁶, or R⁶ and R⁷, taken together, may form a fused ring; R⁸ is a hydrocarbon group; and the symbol * is an asymmetric carbon atom), which comprises reacting an amine of formula (4): 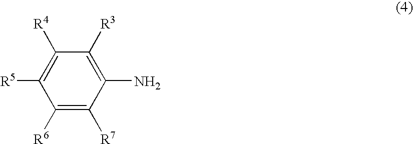
(wherein R³ is hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R³ and R⁴, R⁴ and R⁵, R⁵ and R⁶, or R⁶ and R⁷, taken together, may form a fused ring, provided that either of R³ or R⁷ is hydrogen; and R⁴ to R⁷ are each the same as mentioned above) with an aldehyde of formula (5):
R²—CHO (5)
(wherein R² is the same as mentioned above), and reacting the resulting imine with a N-vinyl carbamate of formula (3): 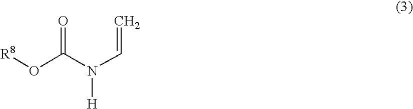
(wherein R⁸ is the same as mentioned above) in the presence of a chiral Lewis acid.

9) A mixture of an optically active amine of formula (9): 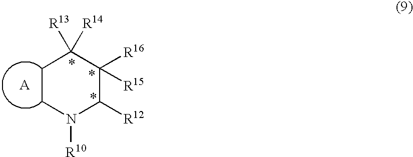
(wherein R¹⁰ is hydrogen, alkyl, aryl, substituted alkyl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹² is hydrocarbon, substituted hydrocarbon, COOR¹⁹ (R¹⁹ is a hydrocarbon group or a substituted hydrocarbon group), COR²⁰ (R²⁰ is a substituted amino group) or substituted amino; R¹³, R¹⁵ and R¹⁶ are each independently hydrogen or alkyl; R¹⁴ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, heteroaryloxy, substituted heteroaryloxy, alkylthio, substituted alkylthio, arylthio, substituted arylthio, aralkylthio, substituted aralkylthio, heteroarylthio, substituted heteroarylthio, substituted amino, substituted silyl, alkylseleno, aralkylseleno, arylseleno or heteroarylseleno; and R¹⁶, taken together, may form a ring; the group represented by ring A is aryl, substituted aryl, aromatic heterocyclic group or substituted aromatic heterocyclic group; 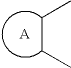
is a divalent group corresponding to the group represented by ring A as mentioned above; and the symbol * is an asymmetric carbon atom) and an optically active compound of formula (9b): 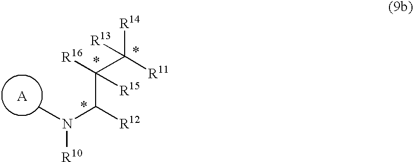
(wherein R¹¹ is a leaving group; the group represented by ring A, R¹⁰, R¹² to R¹⁶ and the symbol * are each the same as mentioned above).

10) A mixture of optically active amines of formula (10): 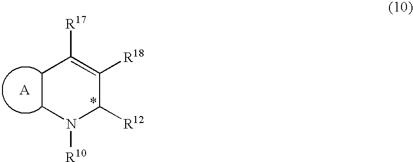
(wherein the group represented by ring A is aryl, substituted aryl, aromatic heterocyclic group or substituted aromatic heterocyclic group; R¹⁰ is hydrogen, alkyl, aryl, substituted alkyl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹² is hydrocarbon, substituted hydrocarbon, COOR¹⁹ (R¹⁹ is a hydrocarbon group or a substituted hydrocarbon group), COR²⁰ (R²⁰ is a substituted amino group) or substituted amino; R¹⁷ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, substituted amino or substituted silyl; R¹⁸ is hydrogen or alkyl; the symbol * is an asymmetric carbon atom; and 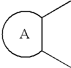
is a divalent group corresponding to the group represented by ring A as mentioned above) and an optically active compound (10b): 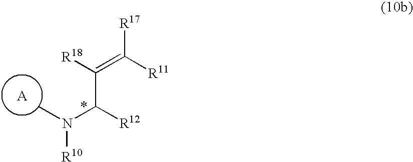
(wherein R¹¹ is a leaving group; R¹⁰, R¹², R¹⁷, R¹⁸, the symbol * and the group represented by ring A are each the same as mentioned above).

11) An optically active compound of formula (9c): 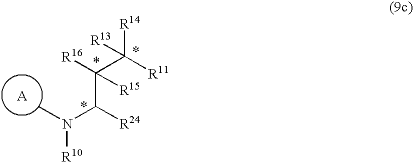
(wherein the group represented by ring A is aryl, substituted aryl, aromatic heterocyclic group or substituted aromatic heterocyclic group; R¹⁰ is hydrogen, alkyl, substituted alkyl, aryl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹¹ is a leaving group; R²⁴ is hydrocarbon, substituted hydrocarbon or COOR¹⁹ (R¹⁹ is a hydrocarbon group or a substituted hydrocarbon group), COR²⁰ (R²⁰ is a substituted amino group) or substituted amino; R¹³, R¹⁵ and R¹⁶ are each independently hydrogen or alkyl; R¹⁴ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, heteroaryloxy, substituted heteroaryloxy, alkylthio, substituted alkylthio, arylthio, substituted arylthio, aralkylthio, substituted aralkylthio, heteroarylthio, substituted heteroarylthio, substituted amino, substituted silyl, alkylseleno, aralkylseleno, arylseleno or heteroarylseleno; R¹⁴ and R¹⁶, taken together, may form a ring; the symbol * is an asymmetric carbon).

12) An optically active compound of formula (10c): 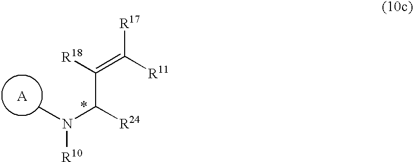
(wherein the group represented by ring A is aryl, substituted aryl, aromatic heterocyclic group or substituted aromatic heterocyclic group; R¹⁰ is hydrogen, alkyl, substituted alkyl, aryl, substituted aryl, acyl, substituted acyl, alkyloxycarbonyl, substituted alkyloxycarbonyl, aralkyloxycarbonyl, substituted aryloxycarbonyl, aralkyloxycarbonyl or substituted aralkyloxycarbonyl; R¹¹ is a leaving group; R²⁴ is hydrocarbon, substituted hydrocarbon or COOR¹⁹ (R¹⁹ is a hydrocarbon group or a substituted hydrocarbon group), COR²⁰ (R²⁰ is a substituted amino group) or substituted amino; R¹⁷ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, substituted amino or substituted silyl; R¹⁸ is hydrogen or alkyl; the symbol * is an asymmetric carbon), provided that when R¹⁵ and R¹⁶ are each the same, the carbon atom to which R¹⁵ and R¹⁶ bind is not an asymmetric carbon atom.

13) The optically active compound of formula (9c) according to claim 11, wherein the optically active compound of formula (9c) is an optically active compound of following formula: 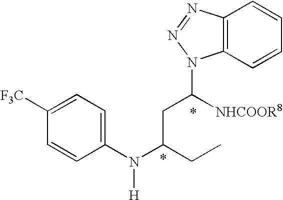
(wherein R⁸ and the symbol * are each the same as mentioned above).

It is an object in accordance with the present invention to provide a method for producing highly optically pure amines, especially tetrahydroquinolines and dihydroquinolines with high optical purity, which method does not require additional procedures such as introduction and removal of protecting groups, and thus has high operability. Also, the method of the present invention is characterized by using a chiral Lewis acid. Therefore, the present invention has an effect that the desired optically active tetrahydroquinolines and dihydroquinolines from the starting amines can be obtained through shorter steps compared with the known common methods.

### BEST MODE FOR CARRYING OUT THE INVENTION

The following is the explanation of the functional groups in the formulae as mentioned above.

The leaving groups represented by R¹¹ and R¹ are those which act in order to produce optically active amines, especially optically active tetrahydroquinolines and dihydroquinolines by reacting an imine equivalent of the above formula (6) (hereinafter called imine equivalent (6)) or an imine equivalent of the above formula (2) (hereinafter called imine equivalent (2)) with an alkene or an alkyne in the presence of a chiral catalyst, whereby such leaving groups are eliminated. Specific examples of the leaving groups include, for example, heterocyclic group such as aliphatic heterocyclic group and aromatic heterocyclic group, acyloxy, halogen, alkoxy, aryloxy, aralkyloxy, heteroaryloxy, alkylthio, arylthio, aralkylthio, heteroarylthio, substituted alkoxy, substituted aryloxy, substituted aralkyloxy, substituted alkylthio, substituted arylthio, substituted aralkylthio, nitro, sulfonyl substituted aliphatic heterocyclic group, substituted aromatic heterocyclic group, substituted heteroaryloxy, substituted heteroarylthio, an onium salt group of nitrogen-containing heteroaromatic compounds, etc. The present:invention can be carried out using these leaving groups, because they are hitherto well established.

The heterocycic groups as the leaving group mentioned above will be hereinafter described in detail.

The acyloxy groups as the above-mentioned leaving group may be of straight or branched ones, including, for example, acyloxy derived from carboxylic acids of 2 to 18 carbon atoms, such as aliphalic carboxylic acids and aromatic carboxylic acids, etc. Specific examples of the acyloxy group include acetoxy, propionyloxy, butyryloxy, pivaloyloxy, pentanoyloxy, hexanoyloxy group, lauroyloxy, stearoyloxy, benzoyloxy, trichloroacetoxy, etc.

Specific examples of the halogen as the leaving group mentioned above include, for example, fluorine atom, chlorine atom, bromine atom, and iodine atom.

The alkoxy groups as the leaving group mentioned above may be of linear, branched or cyclic ones of 1 to 6 carbon atoms, including, for example, methoxy, ethoxy, n-propoxy, 2-propoxy, n-butoxy, 2-butoxy, isobutoxy, tert-butoxy, n-pentyloxy, 2-methylbutoxy, 3-methylbutoxy, 2,2-dimethylpropyloxy, n-hexyloxy, 2-methylpentyloxy, 3-methylpentyloxy, 4-methylpentyloxy, 5-methylpentyloxy, cyclohexyloxy, etc.

The aryloxy groups as the leaving group mentioned above are those having 6 to 14 carbon atoms, including, for example, phenyloxy, naphthyloxy, anthryloxy group, etc.

The aralkyloxy groups as the leaving group mentioned above include aralkyloxy group and substituted aralkyloxy group. The aralkyloxy groups are those having 7 to 12 carbon atoms. Specific examples of such aralkyloxy group include, for example, benzyloxy, 2-phenylethoxy, 1-phenypropoxy, 2-phenylpropoxy, 3-phenylpropoxy, 1-phenylbutoxy, 2-phenylbutoxy, 3-phenylbutoxy, 4-phenylbutoxy, 1-phenylpentyloxy, 2-phenylpentyloxy, 3-phenylpentyloxy, 4-phenylpentyloxy, 5-phenylpentyloxy, 1-phenylhexyloxy, 2-phenylhexyloxy, 3-phenylhexyloxy, 4-phenylhexyloxy, 5-phenylhexyloxy, 6-phenylhexyloxy, etc.

The heteroaryloxy groups as the leaving group mentioned above include, for example, ones having 2 to 14 carbon atoms and containing at least one hetero atom, preferably 1 to 3 hetero atom(s) such as nitrogen, oxygen or sulfur. Specific examples of the heteroaryloxy group include 2-pyridyloxy, 2-pyrazyloxy, 2-pyrimidyloxy, 2-quinolyloxy, etc.

The aliphatic heterocyclic groups as the leaving group mentioned above include, for example, ones of 2 to 14 carbon atoms. The aliphatic heterocyclic groups include, for example, five- to eight-membered, preferably five- or six-membered, monocyclic, polycyclic or fused aliphatic heterocyclic groups, which may contain at least one hetero atom, preferably 1 to 3 hetero atom(s) such as nitrogen, oxygen and/or sulfur. Specific examples of the aliphatic heterocyclic group are pyrrolidyl-2-on, piperidino, piperadinyl, morpholino, morpholinyl, tetrahydrofuryl, tetrahydropyranyl, etc.

The aromatic heterocyclic groups as the leaving group mentioned above include, for example, ones of 2 to 15 carbon atoms. The aromatic heterocyclic groups include, for example, five- to eight-membered, preferably five- or six-membered, monocyclic, polycyclic or fused heteroaryl groups, which may contain at least one hetero atom, preferably 1 to 3 hetero atom(s) such as nitrogen, oxygen or sulfur. Specific examples of the aromatic heterocyclic groups are furyl, thienyl, pyridyl, pyrimidyl, pyrazyl, pyridazyl, pyrazolyl, imidazolyl, oxazolyl, thiazolyl, benzofuryl, benzothienyl, quinolyl, isoquinolyl, quinoxalyl, phthalazyl, quinazolyl, naphthyridyl, cinnolyl, benzimidazolyl, benzoxazolyl, benzothiazolyl group, etc.

The alkylthio groups as the leaving group mentioned above include linear, branched or cyclic ones of 1 to 6 carbon atoms. Specific examples of the alkylthio group are methylthio, ethylthio, n-propylthio, 2-propylthio, n-butylthio, 2-butylthio, isobutylthio, tert-butylthio, pentylthio, hexylthio, cyclohexylthio, etc.

The arylthio groups as the leaving group mentioned above are, for example, ones of 6 to 14 carbon atoms, including phenylthio, naphthylthio, etc.

The aralkylthio groups as the leaving group mentioned above include, for example, ones of 7 to 12 carbon atoms, including benzylthio, 2-phenethylthio, etc.

The heteroarylthio groups as the leaving group mentioned above include, for example, ones of 2 to 14 carbon atoms, which may contain at least one hetero atom, preferably 1 to 3 hetero atom(s) such as nitrogen, oxygen or sulfur. Specific examples of the heteroarylthio group include, for example, 2-pyridylthio, 2-benzimidazolylthio, 2-benzoxazolylthio, 2-benzothiazolylthio, 4-nitrophenylthio, 2-nitrophenylthio, etc.

The sulfonyl groups as the leaving group mentioned above include, for example, a substituted sulfonyl group, such as alkylsulfonyl, substituted alkylsulfonyl, arylsulfonyl and substituted arylsulfonyl, represented by R^{b}—SO₂— (R^{b} is a hydrocarbon group, a substituted hydrocarbon group or a substituted amino group). Specific examples of such sulfonyl group include methanesulfonyl, trifluoromethanesulfonyl, phenylsulfonyl, p-toluenesulfonyl, —SO₂N(CH₃)₂, or the like. The hydrocarbon group, substituted hydrocarbon group and substituted amino group, represented by R^{b}, have each the same meaning as defined for the hydrocarbon group and substituted hydrocarbon group which are mentioned above as a substituent.

With respect to the substituted amino group as a substituent in said substituted aryl group or substituted aromatic heterocyclic group, specific examples of the amino group substituted by alkyl, namely, alkylamino groups include mono or dialkylamino such as N-methylamino, N,N-dimethylamino, N,N-diethylamino, N,N-diisopropylamino, N-cyclohexylamino, etc. Specific examples of the amino group substituted by an aryl group, namely, arylamino includes mono or diarylamino such as N-phenylamino, N,N-diphenylamino, N-naphthylamino, N-naphthyl-N-phenylamino, etc. Specific examples of the amino group substituted by an aralkyl group, namely, aralkylamino include mono- or di-aralkylamino such as N-benzylamino, N, N-dibenzylamino, etc. Specific examples of the amino group substituted by an acyl group, namely, acylamino, include formylamino, acetylamino, propionylamino, pivaloylamino, pentanoylamino, hexanoylamino, benzoylamino, etc. Specific examples of the amino group substituted by an alkoxycarbonyl group, namely, alkoxycarbonylamino, include methoxycarbonylamino, ethoxycarbonylamino, n-propoxycarbonylamino, n-butoxycarbonylamino, tert-butoxycarbonylamino, pentyloxycarbonylamino, hexyloxycarbonylamino, etc. Amino groups substituted by an aryloxycarbonyl group, namely, aryloxycarbonylamino, includes, for example, an amino group in which one hydrogen atom of such amino group is substituted by said aryloxycarbonyl group, and specific examples of such amino group include phenoxycarbonylamino, naphthyloxycarbonylamino, etc. Specific examples of the amino group substituted by aralkyloxycarbonyl group, namely, aralkyloxycarbonylamino include benzyloxycarbonylamino or the like. Specific examples of the sulfonylamino group include —NHSO₂CH₃, —NHSO₂C₆H₅, —NHSO₂C₆H₄CH₃, —NHSO₂CF₃, —NHSO₂N(CH₃)₂, etc.

The substituted alkoxy groups as the leaving group mentioned above include an alkoxy group in which at least one hydrogen atom is substituted by a substituent such as halogenated hydrocarbon, alkoxy, halogen, amino, substituted amino or alkylendioxy. The alkoxy group and halogen atom are each the same as mentioned above. Further, the substituted amino group and alkylendioxy group may have each the same meaning as those which are a substituent in the substituted aryl groups or substituted aromatic heterocyclic groups mentioned below. Specific examples of the alkoxy group substituted by alkoxy group include methoxymethoxy, ethoxyethoxy, methoxyethoxy, etc.

The substituted aryloxy groups as the leaving group mentioned above include an aryloxy group in which at least one hydrogen atom is substituted by a substituent such as alkyl, halogenated hydrocarbon, alkoxy, halogen, amino and substituted amino, and an aryloxy group in which two adjacent hydrogen atoms are substituted by alkylendioxy. Specific examples of the substituted aryloxy group include 4-nitrophenyloxy, 2-nitrophenyloxy, etc.

The substituted aralkyloxy groups as the leaving group mentioned above include an aralkyloxy group in which at least one hydrogen atom is substituted by a substituent such as alkyl, halogenated hydrocarbon, alkoxy, halogen, amino and substituted amino, and an aralkyloxy group in which two adjacent hydrogen atoms are substituted by alkylendioxy.

The substituted alkylthio groups as the leaving group mentioned above include an alkylthio group, in which at least one hydrogen atom is substituted by a substituent such as alkyl, halogenated hydrocarbon, alkoxy, halogen, amino, substituted amino, nitro and alkylendioxy.

The substituted arylthio groups as the leaving group mentioned above include an arylthio group, in which at least one hydrogen atom is substituted by a substituent such as alkyl, halogenated hydrocarbon, alkoxy, halogen, amino, substituted amino, nitro and alkylendioxy. Specific examples of the substituted arylthio group include 4-nitrophenylthio, 2-nitrophenylthio, etc.

The substituted aralkylthio groups as the leaving group mentioned above include an aralkylthio group, in which at least one hydrogen atom is substituted by a substituent such as alkyl, halogenated hydrocarbon, alkoxy, halogen, amino, substituted amino and nitro.

The substituted aliphatic heterocyclic groups as the leaving group mentioned above include an aliphatic heterocyclic group, in which at least one hydrogen atom is substituted by a substituent such as alkyl, halogenated hydrocarbon, alkoxy, halogen, amino and substituted amino.

The substituted aromatic heterocyclic groups as the leaving group mentioned above include an aromatic heterocyclic group, in which at least one hydrogen atom is substituted by a substituent such as alkyl, halogenated hydrocarbon, alkoxy, halogen, amino and substituted amino.

The substituted heteroaryloxy groups as the leaving group mentioned above include a heteroaryloxy group, in which at least one hydrogen atom is substituted by a substituent such as alkyl, halogenated hydrocarbon, alkoxy, halogen, amino and substituted amino.

The substituted heteroarylthio groups as the leaving group mentioned above include a heteroarylthio group, in which at least one hydrogen atom is substituted by a substituent such as alkyl, halogenated hydrocarbon, alkoxy, halogen, amino and substituted amino group.

Specific examples of the onium salt group of nitrogen-containing heteroaromatic compounds as the leaving group mentioned above include groups of the following formulae: 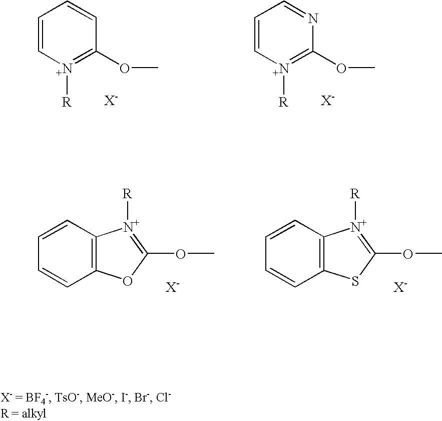
(wherein Ts is p-toluensulfonyl and Vs is methanesulfonyl represented by Ye; alkyl is alkyl represented by R; hereinafter the same)

Specific examples of the heterocyclic group as the leaving group mentioned above include said aliphalic heterocyclic group, said aromatic heterocyclic group, said substituted aliphalic heterocyclic group, said substituted aromatic heterocyclic group, onium salt group of nitrogen-containing aromatic compounds mentioned above, and groups represented by the following formulae, among which the heterocyclic groups represented by the following formulae are preferable.

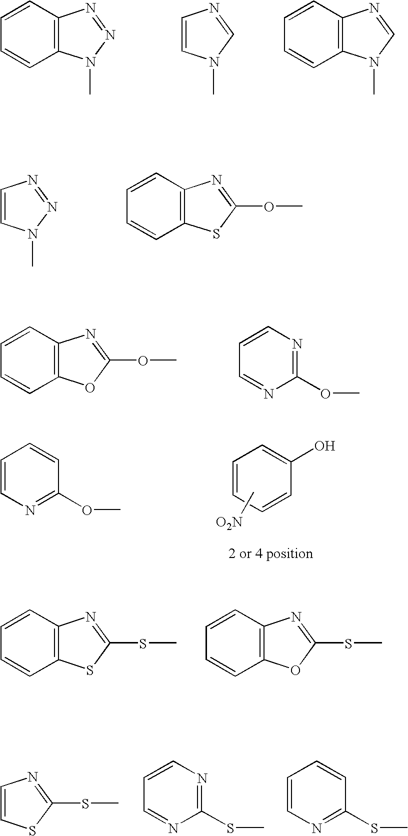

Among those leaving groups, heterocyclic group, acyloxy group, alkoxy group, aryloxy group, heteroaryloxy group, aromatic heterocyclic group, alkylthio group, arylthio group, heteroarylthio group, substituted heteroaryloxy group, substituted heteroarylthio group, and onium salts group of nitrogen-containing heteroaromatic compounds are preferable.

The groups represented by ring A in formula (6) and other formulae are aryl, substituted aryl, aromatic heterocyclic group or substituted aromatic heterocyclic group. The aryl group represented by ring A includes, for example, aryl of 6 to 14 carbon atoms, and specific examples include phenyl, naphthyl, etc.

The substituted aryl groups include an aryl group, in which at least one hydrogen atom of the aryl group mentioned above is substituted by a substituent, and an aryl group, in which two adjacent hydrogen atoms are substituted by a substituent such as alkylenedioxy, etc. With respect to the substituents, they will be hereinafter described.

The aromatic heterocyclic groups as the group represented by ring A include, for example, five- to eight-membered, preferably five- or six-membered, monocyclic, polycyclic or fused cyclic heteroaryl groups which have 2 to 15 carbon atoms and contain at least one hetero atom, preferably 1 to 3 hetero atom(s) such as nitrogen, oxygen or sulfur. Specific examples of such aromatic heterocyclic groups include furyl, thienyl, pyridyl, pyrimidyl, pyrazyl, pyridazyl, pyrazoryl, imidazolyl, oxazolyl, thiazolyl, benzofuryl, benzothienyl, quinolyl, isoquinolyl, quinoxalyl, phthalazyl, quinazolyl, naphthyridyl, cinnolyl, benzimidazolyl, benzoxazolyl, benzthiazolyl or the like.

The substituted aromatic heterocyclic groups as the group represented by ring A include an aromatic heterocyclic group, in which at least one hydrogen atom is substituted by a substituent.

Specific examples of substituents in the substituted aryl or substituted aromatic heterocyclic group mentioned above include hydrocarbon, substituted hydrocarbon, halogen, halogenated hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, alkylthio, substituted alkylthio, arylthio, substituted arylthio, aralkylthio, substituted aralkylthio, acyl, substituted acyl, acyloxy, alkyloxycarbonyl, substituted alkyloxycarbonyl, aryloxycarbonyl, substituted aryloxycarbonyl, aralkyloxycarbonyl, substituted aralkyloxycarbonyl, alkylenedioxy, hydroxy, nitro, amino, substituted amino, cyano, carboxyl, sulfo, sulfonyl, substituted silyl, etc.

The halogen, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, alkylthio, substituted alkylthio, arylthio, substituted arylthio, aralkylthio, substituted aralkylthio and acyloxy are each the same as those represented by ring A or as the leaving group mentioned above.

Specific examples of the hydrocarbon group as a substituent include alkyl, alkenyl, alkynyl, aryl, aralkyl, etc.

The alkyl groups may be of linear or branched ones of 1 to 10 carbon atoms, preferably 1 to 6 carbon atoms. Specific examples of the alkyl group include methyl, ethyl, n-propyl, 2-propyl, n-butyl, 2-butyl, isobutyl, tert-butyl, n-pentyl, 2-pentyl, tert-pentyl, 2-methylbutyl, 3-methylbutyl, 2,2-dimethylpropyl, n-hexyl, 2-hexyl, 3-hexyl, 2-methylpentan-2-yl, 3-methylpentan-3-yl, 2-methylpentyl, 3-methylpentyl, 4-methylpentyl, 2-methylpentan-3-yl, heptyl, octyl, 2-ethylhexyl, nonyl, decyl, cyclopropyl, cyclobutyl, cyclopentyl, cyclohexyl, etc.

The alkenyl groups may be of linear or branched ones of 2 to 10 carbon atoms, preferably 2 to 6 carbon atoms. Specific examples of the alkenyl group include ethenyl, propenyl, 1-butenyl, 2- butenyl, pentenyl, hexenyl, heptenyl, octenyl, nonenyl, decenyl, etc.

The alkynyl groups may be of linear or branched ones of 2 to 10 carbon atoms, preferably 2 to 6 carbon atoms. Specific examples of the alkynyl group include ethynyl, 1-propynyl, 2-propynyl, 1-butynyl, 3-butynyl, pentynyl, hexynyl, etc.

The aryl groups include, for example, ones of 6 to 14 carbon atoms. Specific examples of the aryl group include phenyl, naphtyl, anthryl, biphenyl, etc.

The aralkyl groups include, for example, ones of preferably 7 to 12 carbon atoms, in which at least one hydrogen atom of the alkyl moiety is substituted by said aryl group. Specific examples of the aralkyl group include benzyl, 2-phenylethyl, 1-phenylpropyl, 3-naphtylpropyl, etc.

The halogenated hydrocarbon groups as a substituent include a group, in which at least one hydrogen atom of the said hydrocarbon group is halogenated (for example, by fluorination, chlorination, bromination or iodination). Specific examples of such halogenated hydrocarbon group include, for example, halogenated alkyl. The halogenated alkyl groups include, for example, ones of 1 to 10 carbon atom, and specific examples of such halogenated hydrocarbon groups include chloromethyl, bromomethyl, 2-chloroethyl, 3-bromopropyl, fluoromethyl, fluoroethyl, fluoropropyl, fluorobutyl, fluoropentyl, fluorohexyl, fluoroheptyl, fluorooctyl, fluorononyl, fluorodecyl, difluoromethyl, difluoroethyl, fluorocyclohexyl, trifluoromethyl, 2,2,2-trifluoroethyl, 3,3,3-trifluoropropyl, pentafluoroethyl, 3,3,4,4,4-pentafluorobutyl, perfluoro-n-propyl, perfluoroisopropyl, perfluoro-n-butyl, perfluoroisobutyl, perfluoro-tert-butyl, perfluoro-sec-butyl, perfluoropentyl, perfluoroisopentyl, perfluoro-tert-pentyl, perfluoro-n-hexyl, perfluoroisohexyl, perfluoroheptyl, perfluorooctyl, perfluorononyl, perfluorodecyl, 2-perfluorooctylethyl, perfluorocyclopropyl, perfluorocyclopentyl, perfluorocyclohexyl, etc. Among those halogenated alkyl groups, the halogenated alkyl groups of 1 to 6 carbon atoms are preferable, more preferably of 1 to 3 carbon atoms, especially preferably the fluorine-containing alkyl groups of 1 to 3 carbon atoms such as fluoromethyl, fluoroethyl, fluoropropyl, difluoromethyl, difluoroethyl, trifluoromethyl, 2,2,2-trifluoroethyl, 3,3,3-trifluoropropyl, pentafluoroethyl, perfluoro-n-propyl, perfluoroisopropyl, etc.

The acyl groups as a substituent may be of straight or branched ones of 1 to 18 carbon atoms, and specific examples of such acyl group include formyl, acetyl, propionyl, butyryl, pivaloyl, pentanoyl, hexanoyl, lauroyl, stearoyl, benzoyl, etc.

The alkyloxycarbonyl groups as a substituent may be of straight or branched ones of 2 to 19 carbon atoms, and specific examples of such alkyloxycarbonyl group include methoxycarbonyl, ethoxycarbonyl, n-propoxycarbonyl, 2-propoxycarbonyl, 2-butoxycarbonyl, tert-butoxycarbonyl, pentyloxycarbonyl, hexyloxycarbonyl, 2-ethylhexyloxycarbonyl, lauryloxycarbonyl, stearyloxycarbonyl, cyclohexyloxycarbonyl, etc.

The aryloxycarbonyl groups as a substituent include ones of 7 to 20 carbon atoms, and specific examples of such aryloxycarbonyl group include phenoxycarbonyl, naphthyloxycarbonyl, etc.

The aralkyloxycarbonyl groups as a substituent include ones of 8 to 15 carbon atoms, and specific examples of such aralkyloxycarbonyl group include benzyloxycarbonyl, phenylethoxycarbonyl, 9-fluorenylmethyloxycarbonyl, etc.

The alkylenedioxy groups as a substituent include ones of 1 to 3 carbon atoms, and specific examples of such alkylenedioxy group include methylenedioxy, ethylenedioxy, propylenedioxy, trimethylenedioxy, etc.

The substituted hydrocarbon groups as a substituent include a hydrocarbon group, in which at least one hydrogen atom of said hydrocarbon group is substituted by the above-mentioned substituent, and specific examples of such substituted hydrocarbon group include substituted alkyl, substituted alkenyl, substituted alkynyl, substituted aryl, substituted aralkyl, etc.

The substituted alkyl groups in said substituted hydrocarbon group include an alkyl group, in which at least one hydrogen atom of said alkyl group is substituted by the above-mentioned substituent. Alkyl groups substituted by halogen atom, namely, halogenated alkyl groups are the same as the halogenated alkyl group in the halogenated hydrocarbon group described as a substituent.

The substituents in the substituted alkenyl group (for example, substituted vinyl) or substituted alkynyl (for example, substituted propargyl) may be the same as those in said substituted alkyl group.

The substituted aryl groups include an aryl group, in which at least one hydrogen atom is substituted by said substituent such as hydrocarbon, halogen, halogenated hydrocarbon, aliphatic heterocyclic group, aromatic heterocyclic group, alkoxy, aralkyloxy, aryloxy, acyl, acyloxy, alkyloxycarbonyl, aryloxycarbonyl, aralkyloxycarbonyl, hydroxy, nitro, amino, and substituted amino, and an aryl group in which two adjacent hydrogen atoms of said aryl group are substituted by an alkylenedioxy group. Specific examples of the aryl group substituted by an alkyl group include tolyl, xylyl, mesityl, etc.

The substituted aralkyl groups include an aralkyl group, in which at least one hydrogen atom of the aryl moiety in said aralkyl group is substituted by said substituent such as hydrocarbon, halogen, halogenated hydrocarbon, aliphatic heterocyclic group, aromatic heterocyclic group, alkoxy, aralkyloxy, aryloxy, acyl, acyloxy, alkyloxycarbonyl, aryloxycarbonyl, aralkyloxycarbonyl, hydroxy, nitro, amino, substituted amino group, and an aralkyl group in which two adjacent hydrogen atoms of the aryl moiety in said aralkyl group are substituted by a substituent such as an alkylenedioxy group or the like.

The substituted acyl groups as a substituent in said substituted aryl or substituted aromatic heterocyclic group include an acyl group, in which at least one hydrogen atom of said acyl is substituted by a substituent such as alkyl, halogenated hydrocarbon group, alkoxy, halogen atom, amino, substituted amino, nitro, alkylenedioxy, etc.

The substituted alkyloxycarbonyl groups as a substituent in said substituted aryl or substituted aromatic heterocyclic group include an alkyloxycarbonyl group, in which at least one hydrogen atom of said alkyloxycarbonyl is substituted by a substituent such as alkyl, halogenated hydrocarbon, alkoxy, halogen, amino, substituted amino, nitro, alkylenedioxy, etc.

The substituted aryloxycarbonyl groups as a substituent in said substituted aryl or substituted aromatic heterocyclic group include an aryloxycarbonyl group, in which at least one hydrogen atom of said aryloxycarbonyl is substituted by a substituent such as alkyl, halogenated hydrocarbon, alkoxy, halogen, amino, substituted amino, nitro, or the like, and an aryloxycarbonyl group in which two adjacent hydrogen atoms of the aryl moiety in said aryloxycarbonyl are substituted by an alkylenedioxy group or the like.

The substituted aralkyloxycarbonyl groups as a substituent in said substituted aryl or substituted aromatic heterocyclic group include an aralkyloxycarbonyl group, in which at least one hydrogen atom of said aralkyloxycarbonyl group is substituted by a substituent such as alkyl, halogenated hydrocarbon, alkoxy, halogen, amino, substituted amino, nitro or the like, and an aralkyloxycarbonyl group in which two adjacent hydrogen atoms of the aryl moiety in said aralkyloxycarbonyl are substituted by an alkylenedioxy group or the like.

The substituted amino groups as a substituent in said substituted aryl or substituted aromatic heterocyclic group include an amino group, in which one or two hydrogen atoms of the amino group is substituted by a substituent such as a protecting group or the like. As such protecting group, any group which can be used as amino-protecting groups is employable. The amino-protecting groups include, for example, those described as amino-protecting groups in “PROTECTIVE GROUPS IN ORGANIC SYNTHESIS Third Edition (JOHN WILEY & SONS, INC.)”. Specific examples of such amino protecting group include alkyl, aryl, aralkyl, acyl, alkyloxycarbonyl, aryloxycarbonyl, aralkyloxycarbonyl, sulfonyl, etc.

The alkyl, aryl, aralkyl, acyl, alkyloxycarbonyl, aryloxycarbonyl, and aralkyloxycarbonyl groups as an amino-protecting group mentioned above have the same meaning as those mentioned above as a substituent.

The sulfonyl groups as said amino-protecting group include, for example, a substituted sulfonyl group, such as alkylsulfonyl, substituted alkylsulfonyl, arylsulfonyl and substituted arylsulfonyl, represented by R^{b}—SO₂— (R^{b} is a hydrocarbon group, a substituted hydrocarbon group or a substituted amino group). Specific examples of such sulfonyl group include methanesulfonyl, trifluoromethanesulfonyl, phenylsulfonyl, p-toluenesulfonyl, —SO₂N(CH₃)₂, or the like. The hydrocarbon group, substituted hydrocarbon group and substituted amino group, represented by R^{b}, have each the same meaning as defined for the hydrocarbon group, substituted hydrocarbon group and substituted amino group which are mentioned above as a substituent.

With respect to the substituted amino group as a substituent in said substituted aryl group or substituted aromatic heterocyclic group, specific examples of the amino group substituted by alkyl, namely, alkylamino groups include mono or dialkylamino such as N-methylamino, N,N-dimethylamino, N,N-diethylamino, N,N-diisopropylamino, N-cyclohexylamino, etc. Specific examples of the amino group substituted by an aryl group, namely, arylamino includes mono or diarylamino such as N-phenylamino, N,N-diphenylamino, N-naphthylamino, N-naphthyl-N-phenylamino, etc. Specific examples of the amino group substituted by an aralkyl group, namely, aralkylamino include mono- or di-aralkylamino such as N-benzylamino, N,N-dibenzylamino, etc. Specific examples of the amino group substituted by an acyl group, namely, acylamino, include formylamino, acetylamino, propionylamino, pivaloylamino, pentanoylamino, hexanoylamino, benzoylamino, etc. Specific examples of the amino group substituted by an alkoxycarbonyl group, namely, alkoxycarbonylamino, include methoxycarbonylamino, ethoxycarbonylamino, n-propoxycarbonylamino, n-butoxycarbonylamino, tert-butoxycarbonylamino, pentyloxycarbonylamino, hexyloxycarbonylamino, etc. Amino groups substituted by an aryloxycarbonyl group, namely, aryloxycarbonylamino, includes, for example, an amino group in which one hydrogen atom of such amino group is substituted by said aryloxycarbonyl group, and specific examples of such amino group include phenoxycarbonylamino, naphthyloxycarbonylamino, etc. Specific examples of the amino group substituted by aralkyloxycarbonyl group, namely, aralkyloxycarbonylamino include benzyloxycarbonylamino or the like. Specific examples of the sulfonylamino group include —NHSO₂CH₃, —NHSO₂C₆H₅, —NHSO₂C₆H₄CH₃, —NHSO₂CF₃, —NHSO₂N(CH₃)₂, etc.

The sulfonyl groups as a substituent in said substituted aryl or substituted aromatic heterocyclic group may have the same meaning as those mentioned above in said amino-protecting group.

The substituted silyl groups as a substituent in said substituted aryl group or substituted aromatic heterocyclic group include, for example, a tri-substituted silyl group, in which three hydrogen atoms of such silyl group are substituted by a substituent such as said alkyl, aryl, aralkyl, alkoxy, etc., and specific examples of the substituted silyl group include trimethylsilyl, tert-butyldimethylsilyl, tert-butyldiphenylsilyl, triphenylsilyl, trimethoxysilyl, triethoxysilyl, etc.

The groups represented by ring A is aryl, substituted aryl, aromatic heterocyclic group or substituted aromatic heterocyclic group, and the aryl group in the groups represented by ring A includes, for example, an aryl group of 6 to 14 carbon atoms, and specific examples of such aryl group include phenyl, naphthyl, etc.

The substituted aryl groups in the groups represented by ring A include an aryl group, in which at least one hydrogen atom of said aryl group is substituted by a substituent such as alkyl, halogenated hydrocarbon, alkoxy, halogen, amino, substituted amino or the like, and an aryl group in which two adjacent hydrogen atoms of said aryl group are substituted by a substituent such as alkylenedioxy or the like. The alkyl, halogenated hydrocarbon, alkoxy, halogen or substituted amino are the same as mentioned above. Specific examples of the aryl group substituted by an alkyl group include tolyl, xylyl or the like. The alkylenedioxy groups include those of 1 to 3 carbon atoms, and specific examples of such alkylenedioxy include methylenedioxy, ethylenedioxy, propylenedioxy, etc.

The aromatic heterocyclic groups in the groups represented by ring A include, for example, five- to eight-membered, preferably five- or six-membered,, monocyclic, polycyclic or fused cyclic heteroaryl groups of 2 to 15 carbon atoms, which may contain at least one hetero atom, preferably 1 to 3 hetero atoms such as nitrogen, oxygen or sulfur. Specific examples of such aromatic heterocyclic group include furyl, thienyl, pyridyl, pyrimidyl, pyrazyl, pyridazyl, pyrazolyl, imidazolyl, oxazolyl, thiazolyl, benzofuryl, benzothienyl, quinolyl, isoquinolyl, quinoxalyl, phthalazyl, quinazolyl, naphthyridyl, cinnolyl, benzimidazolyl, benzoxazolyl, benzthiazolyl, etc.

The substituted aromatic heterocyclic groups in the groups represented by ring A include an aromatic heterocyclic group, in which at least one hydrogen atom of said aromatic heterocyclic group is substituted by a substituent such as alkyl, halogenated hydrocarbon group, alkoxy, halogen atom, etc. The alkyl group, halogenated hydrocarbon group, alkoxy group and halogen atom have each the same meaning as mentioned above.

Alkyl, substituted alkyl, aryl, substituted aryl, acyl, substituted acyl, alkyloxycarbonyl, substituted alkylcarbonyl, aryloxycarbonyl, substituted aryloxycarbonyl, aralkyloxycarbonyl, and substituted aralkyloxycarbonyl represented by R¹⁰ in formula (6) and other formulae have each the same meaning as alkyl, substituted alkyl, aryl, substituted aryl, acyl, substituted acyl group, alkyloxycarbonyl, substituted alkyloxycarbonyl, aryloxycarbonyl, substituted aryloxycarbonyl, aralkyloxycarbonyl, and substituted aralkyloxycarbonyl mentioned above as a substituent in said substituted aryl group or substituted aromatic heterocyclic group.

Hydrocarbon groups and substituted hydrocarbon groups represented by R¹², and hydrocarbon group and substituted hydrocarbon group represented by R¹⁹ in COOR¹⁹ have each the same meaning as those described above as a substituent. Substituted amino groups represented by R¹² and substituted amino groups represented by R²⁰ in COR²⁰have each the same meaning as the substituted amino group described as a substituent. In the present invention, a group represented by R¹² is preferably other than hydrogen atom among groups defined by R¹².

The alkyl groups represented by R¹³, R¹⁵ and R¹⁶ in formula (7) and other formulae have each the same meaning as those in the hydrocarbon group described above as a substituent. The aryl group and substituted aryl group represented by R¹⁴ have each the same meaning as the aryl group in the hydrocarbon group, and the substituted aryl group in the substituted hydrocarbon group described above as a substituent. The aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, heteroaryloxy, substituted heteroaryloxy, alkylthio, substituted alkylthio, aralkylthio, substituted arylthio, heteroarylthio, substituted heteroarylthio and substituted silyl represented by R¹⁴ have each the same meaning as those described as said substituent.

The alkylseleno groups represented by R¹⁴ may be straight, branched or cyclic ones of 1 to 6 carbon atoms. Specific examples of such alkylseleno group include methylseleno, ethylseleno, n-propylseleno, 2-propylseleno, n-butylseleno, 2-butylseleno, isobutylseleno, tert-butylseleno, pentylseleno, hexylseleno, cyclohexylseleno, etc.

The aralkylseleno groups represented by R¹⁴ include, for example, ones of 7 to 12 carbon atoms. Specific examples of such aralkylseleno group include benzylseleno, 2-phenethylseleno, etc.

The arylseleno groups represented by R¹⁴ include, for example, ones of 6 to 14 carbon atoms, and specific examples of such arylseleno group include phenylseleno, naphtylseleno, etc.

The heteroarylseleno groups represented by R¹⁴ include, for example, a heteroaryloxy group of 2 to 14 carbon atoms, which contain at least one hetero atom, preferably, 1 to 3 hetero atom(s) such as nitrogen, oxygen or sulfur, and specific examples of such heteroarylseleno group include 4-pyridylseleno, 2-benzimidazolylseleno, 2-benzoxazolylseleno, 2-benzothiazolylseleno, etc.

The aliphatic heterocyclic groups, substituted aliphatic heterocyclic group, aromatic heterocyclic group and substituted aromatic heterocyclic group will be hereinafter described in detail.

The substituted amino groups represented by R¹⁴ is shown by formula of —NR²¹R²² (R²¹ is hydrogen or alkyl; R²² is alkyl, cyano, alkyloxycarbonyl, aryloxycarbonyl, aralkyloxycarbonyl, acyl, sulfonyl or alkoxy. Alternatively, R²¹ and R²² taken together may form a ring such as cyclic amines or cyclic amides). Specific examples of such cyclic amine and cyclic amide include piperidino, morpholino, pyrrolidino, piperazino, etc.

The alkyl groups represented by R²¹ may be the same as those described above in the substituents.

The alkyl, alkyloxycarbonyl, aryloxycarbonyl, aralkyloxycarbonyl, acyl, sulfonyl, and alkoxy represented by R²² are each the same as those in the substituents described above.

Specific examples of the substituted amino group are the same as those exemplified by the substituted amino groups as a substituent mentioned above.

When R¹⁴ and R¹⁶ in formula (7) and other formulae, taken together, may form a ring, there is exemplified a ring formed by an alkylene chain optionally containing a hetero atom such as oxygen, etc. Specific examples of those rings include 2,3-dihydrofuran, 3,4-dihydro-2H-pyran, etc.

The alkyl groups represented by R¹⁸ in formula (8) and other formulae are the same as those which are a substituent in the hydrocarbon group mentioned above.

The aryl or substituted aryl groups represented by R¹⁷ are the same as those which are a substituent in the hydrocarbon and substituted hydrocarbon group mentioned above. The aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy and substituted silyl groups represented by R¹⁷ are the same as those which are a substituent mentioned above. The substituted amino group represented by R¹⁷ may be the same as the substituted amino group represented by said R¹⁴.

The groups represented by R²³ in formula (7a) and other formulae such as aryl, substituted aryl, aliphatic heterocyclic group, substituted aliphatic heterocyclic group, aromatic heterocyclic group, substituted aromatic heterocyclic group, aryloxy, substituted aryloxy, aralkyloxy, substituted aralkyloxy, heteroaryloxy, substituted heteroaryloxy, alkylthio, substituted alkylthio, aralkylthio, substituted arylthio, heteroarylthio, substituted heteroarylthio, substituted amino, substituted silyl, alkylseleno, aralkylseleno, arylseleno and heteroarylseleno are the same as those described in said R¹⁴.

The aliphatic heterocyclic group, substituted aliphatic heterocyclic group, aromatic heterocyclic group, substituted aromatic heterocyclic group, represented by R¹⁴ and R¹⁷ in formula (7), (7a) and other formulae may have each the same meaning as the aliphatic heterocyclic group, substituted aliphatic heterocyclic group, aromatic heterocyclic group, substituted aromatic heterocyclic group in the above leaving group, respectively. Specific examples of those groups include rings represented by the following formulae.

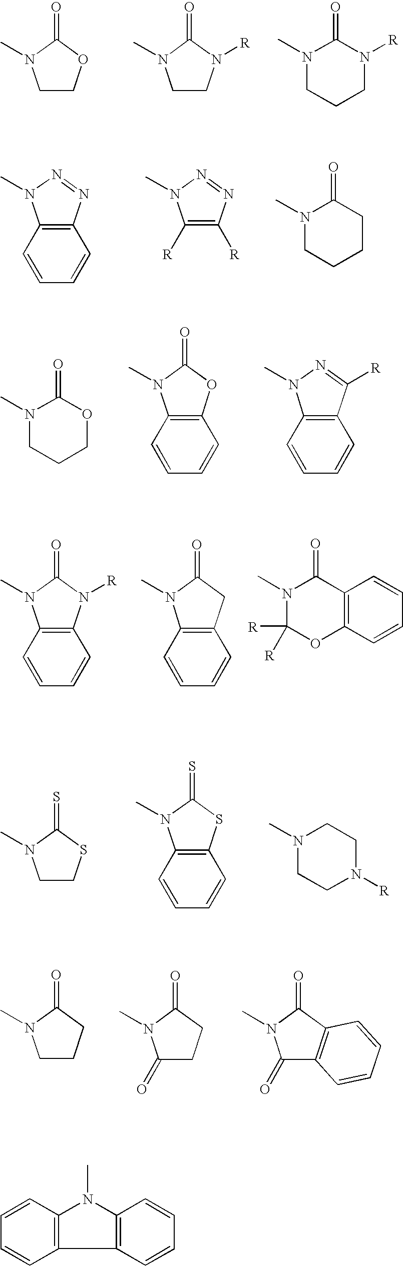

In the above formulae, R is, the same or different, a hydrogen atom or a substituent which is the same as the substituent mentioned above. The said heterocycic group may contain a substituent selected from the above-mentioned various substituents in addition to said R.

The ring which is formed when R²³ and R¹⁶ in formula (7a) and other formulae are taken together may have the same meaning as that which is formed when R¹⁴ and R¹⁶ are taken together.

The hydrocarbon group, halogen atom, halogenated hydrocarbon group, substituted hydrocarbon group, aliphatic heterocyclic group, substituted aliphatic heterocyclic group, aromatic heterocyclic group, substituted aromatic heterocyclic group, alkoxy group, substituted alkoxy group, aralkyloxy group, substituted aralkyloxy group, aryloxy group, substituted aryloxy group, acyl group, acyloxy group, alkyloxycarbonyl group, aryloxycarbonyl group, aralkyloxycarbonyl group, alkylenedioxy group, hydroxy group, nitro group, amino group, substituted amino group, cyano group, carboxyl group, sulfo group, sulfonyl group or substituted silyl group represented by R³ to R⁷ in formula (2) and other formulae are the same as those described as the above substituent.

Here, preferably, R³ is a hydrogen atom. Preferable groups represented by at least one of R⁴ to R⁷ are hydrocarbon group, halogen atom or halogenated hydrocarbon group, and at least one of R⁴ to R⁷ is a halogen atom or halogenated hydrocarbon group, and at least one of R⁴ to R⁷ is more preferably a halogen atom or halogenated alkyl group.

In the formula (2), if it explains more concretely, R³ is preferably a hydrogen atom; and at least one of R⁴ to R⁷ is preferably a hydrogen atom, an alkoxy group, a halogenated hydrocarbon group or a halogen atom; among above, preferably (a) R⁵ is an alkoxy group or a halogenated hydrocarbon group, or (b) at least one of R⁴ to R⁶ is a halogen atom and other R⁴ to R⁷ are each a hydrogen atom; more preferably (c) R⁵ is an alkoxy group or a halogenated hydrocarbon group and R⁴, R⁶, R⁷ are each a hydrogen atom, or (d) R⁴, R⁶ are a halogen atom and R⁵, R⁷ are a hydrogen atom; furthermore preferably (e) R⁵ is a methoxy or a fluorine-containing alkyl group of 1 to 3 carbon atoms and R⁴, R⁶, R⁷ are each a hydrogen atom, or (f) R⁴, R⁶ are each a chlorine atom and R⁵, R⁷ are each a hydrogen atom; the most preferably, R⁵ is a trifluoromethyl and R⁴, R⁶, R⁷ are each a hydrogen atom. These R⁴ to R⁷ are applied to formulae below.

The hydrocarbon group and substituted hydrocarbon group represented by R² in formula (2) and other formulae may have the same meaning as those described as the substituent. The hydrocarbon groups represented by R⁹ in COOR⁹ may have the same meaning as those described as the substituent.

In the formula (2), a hydrocarbon group of R² is preferable, an alkyl group is more preferable, and an ethyl group is furthermore preferable.

The hydrocarbon groups represented by R⁸ in formula (3) and other formulae have the same meaning as those described as the substituent.

In the formula (3), an alkyl group of R⁸ is preferable, and a methyl group is more preferable.

The hydrocarbon group, substituted hydrocarbon group, COOR¹⁹, COR²⁰ and substituted amino group represented by R²⁴ in formula (9c) and (10c) may have the same meaning as each group described in the above R¹².

Plural substituents which are described by the same term, but which are located at different positions from each other or the symbols representing said substituents are different from each other, have each the same meaning, unless otherwise indicated.

Imine equivalents which are represented by formula (6) or formula (2) are referred to as the imine equivalents in the present specification, because these imine equivalents have each the same characteristics and undergo the same behaviors as the above imines, when the above imines are reacted with the above alkenes, especially N-vinyl carbamates or the above alkynes in the presence of chiral catalyst. The imine equivalents represented by formula (6) include, for example, imine equivalent represented by the above formula (2) (hereiafter, referred to as imine equivalent (2)). The leaving group, which are constituents of imine equivalent above, may participate in the reaction. The imine equivalent (2) is preferably an imine equivalent represented by the formula (2-1) mentioned below. 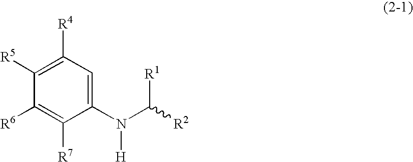
(wherein R¹, R² and R⁴ to R⁷ are each the same as mentioned above) Specific examples of those imine equivalents include N-1-(1-acetyloxy)propyl-4-trifluoromethylaniline, N-1-(1-propionyloxy)propyl-4-trifluoromethylaniline, N-1-(1-butyryloxy))propyl-4-trifluoromethylaniline, and an imine equivalent represented by the following formulae: 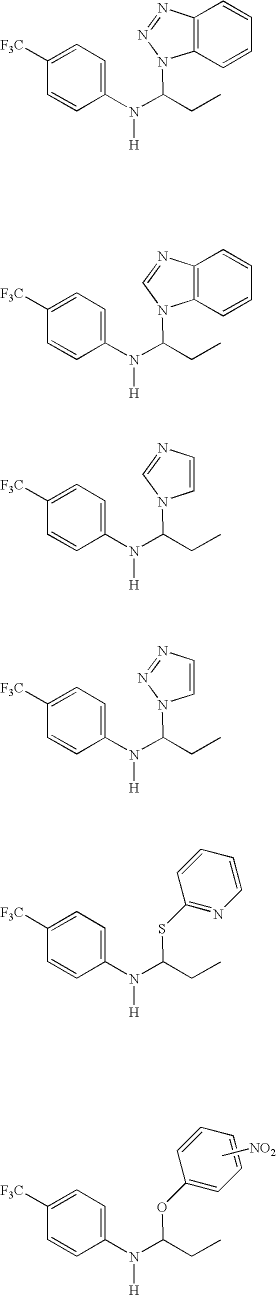 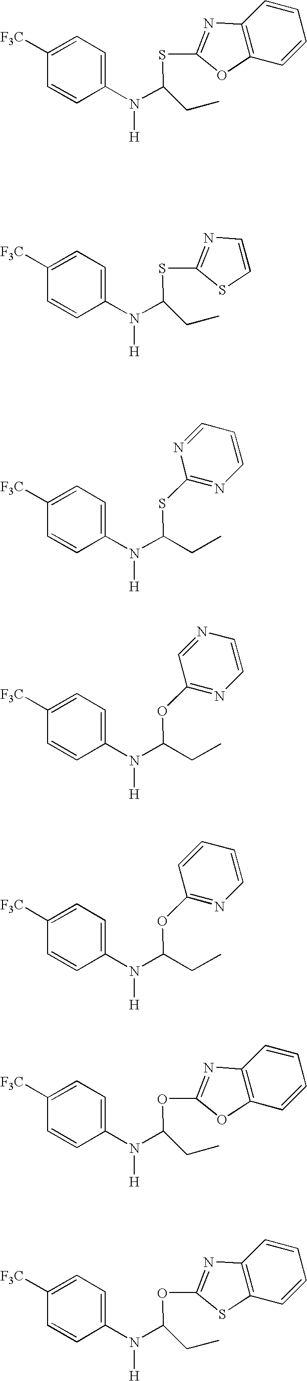 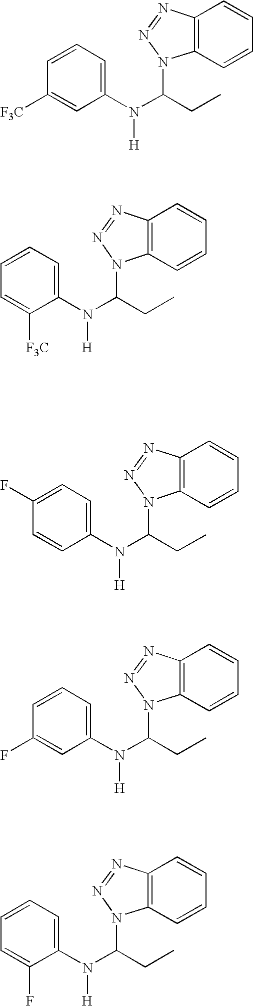 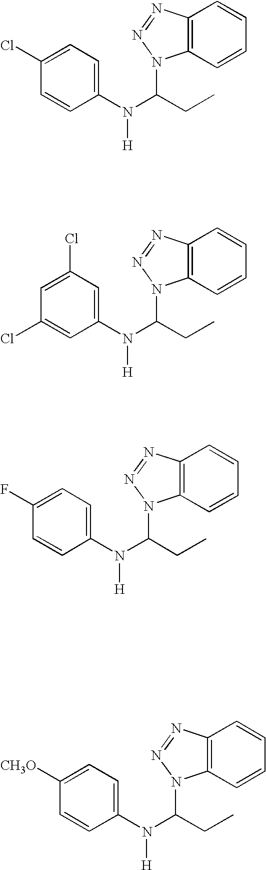
(wherein R is alkyl).

The imines represented by formula (6a) include, for example, an imine represented by formula (2a)(hereinafter referred to as imines (2a)). Among the imines (2a), imines represented by formula (2a-1) as shown below are preferable. 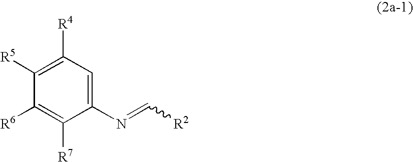
(wherein R² and R⁴ to R⁷ are each the same as mentioned above)

Specific examples of those imines include N-ethylidene-4-trifluoromethylamine, N-propylidene-4-trifluoromethylamine, N-butylidene-4-trifluoromethylamine, N-benzylidene-4-trifluoromethylamine, etc.

The alkenes represented by formula (7) include N-vinyl carbamates (hereinafter referred to as N-vinyl carbamates (3)), cyclopentadiene, 2,3-dihydrofuran, 3,4-dihydro-2H-pyran, N-vinylpyrrolidine, etc., preferably alkenes represented by the above formula (7a), more preferably N-vinyl carbamates (3). Specific examples of the N-vinyl carbamates (3) include methyl N-vinyl carbamate, ethyl N-vinyl carbamate, isopropyl N-vinyl carbamate, butyl N-vinyl carbamate, benzyl N-vinyl carbamate, etc.

The groups represented by R²³ in the above formula (7a) include, preferably aryl, substituted aryl, aliphatic heterocyclic group, substituted aliphatic heterocyclic group, aromatic heterocyclic group, substituted aromatic heterocyclic group, alkylthio, substituted alkylthio, aralkylthio, substituted arylthio, heteroarylthio, substituted heteroarylthio, substituted amino, substituted silyl, alkylseleno, aralkylseleno, arylseleno, heteroarylseleno, etc.

Specific examples of alkynes (8) used in the present invention include compounds represented, for example, by the following formulae:

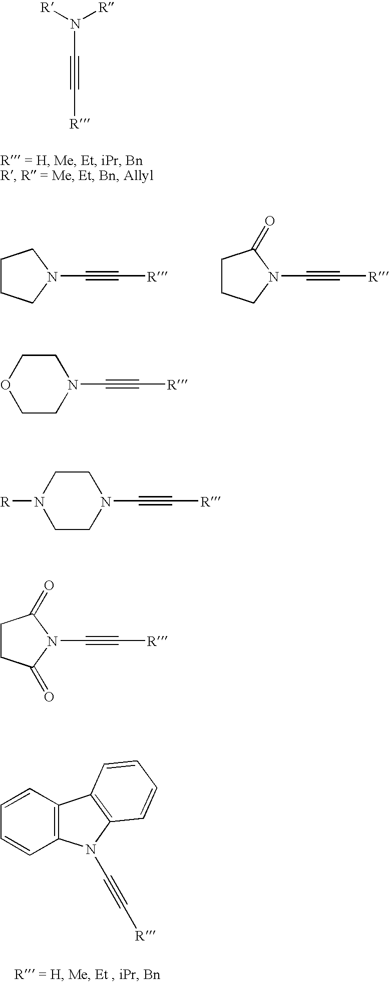

In the above formulae, R is independently a hydrogen atom or a substituent. Me is methyl, Et is ethyl, iPr is isopropyl, Bn is benzyl, and Allyl is allyl. The substituents are each the same as various groups mentioned above. The above heterocycle group may contain a substituent selected from various groups mentioned above in-addition to the above R.

The alkenes (7) and alkynes (8) used in the present invention may be a precursor thereof. Any precursors may be used as long as they act like alkenes (7) or alkynes (8) to give desired optically active amines when the process of the present invention is performed. Specific examples of such precursors to N-vinyl carbamates include bisurethanes represented by the following formula (A) and alkoxy derivatives represented by the following formula (B): 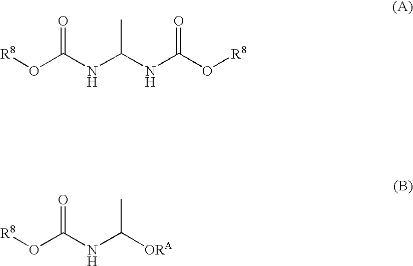
(wherein R^{A} is a hydrocarbon group or a substituted hydrocarbon group, and R⁸ is the same as mentioned above).

The amines (hereinafter referred to as amines(4)) represented by formula (4) are preferably amines represented by formula (4-1): 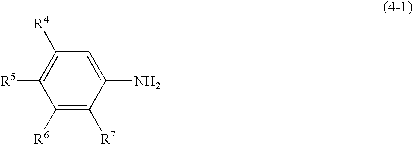
(wherein R⁴ to R7 are each the same as mentioned above). Specific examples of such amines include, for example, 4-trifluoromethylaniline, 3-trifluoromethylaniline, 2-trifluoromethylaniline, 3,5-bis(trifluoromethyl)aniline, 2,5-bis(trifluoromethyl)aniline, 3,4,5-tris(trifluoromethyl)aniline, 4-fluoroaniline, 3-fluoroaniline, 2-fluoroaniline, 3,4-difluoroaniline, 2,4-difluoroaniline, 2,3-difluoroaniline, 3,5-difluoroaniline, 2,3,4-trifluoroaniline, 2,4,5-trifluoroaniline, 4-chloroanline, 3-chloroaniline, 2-chloroaniline, 3,4-dichloroaniline, 3,5-dichloroaniline, 2,3,4-trichloroaniline, 2,4,5-trichloroaniline, 3,4,5-trichloroaniline, 4-bromoaniline, 3-bromoaniline, 2-bromoaniline, 2,4-dibromoaniline, 2,5-dibromoaniline, 3,4,5-tribromoaniline, 4-iodoaniline, 3-todoaniline, 2-iodoaniline, 4-methoxyaniline, 3-methoxyaniline, 2-methoxyaniline etc.

Specific examples of the compounds capable of forming imine equivalents as mentioned above include a hetero compound such as benzotriazole, purine, imidazole, 4-nitrophenol, 2-mercaptopyridine, 2-hydroxypyridine, 2-mercaptobenzothiazole, etc., and an alcohol such as methanol, ethanol, 2-propanol, n-butanol, 2-ethoxyethanol, benzyl alcohol, etc.

The chiral catalyst used in the present invention is a chiral Lewis acid or a compound having such characteristics as the chiral Lewis acid. The chiral Lewis acids are those which are formed from a metal element and a ligand. The metal element includes, for example, a typical element such as boron and aluminium, a transition element such as titanium and zirconium or a rare earth element such as ytterbium, preferably boron, titanium, zirconium or ytterbium, with the proviso that when imines are reacted with alkenes or alkynes, the metal element such as boron, titanium or zirconium except for ytteribium is more preferably used. The ligand includes, for example, ligands shown below. 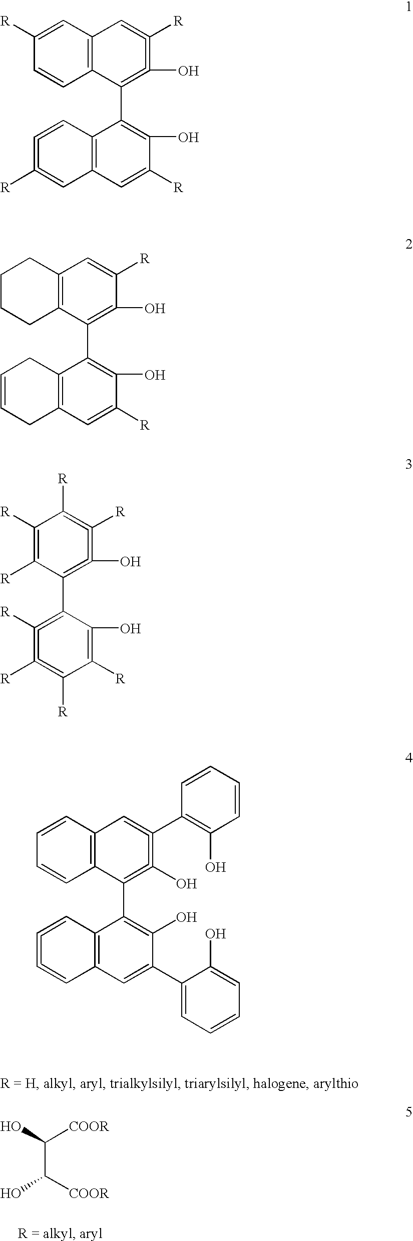 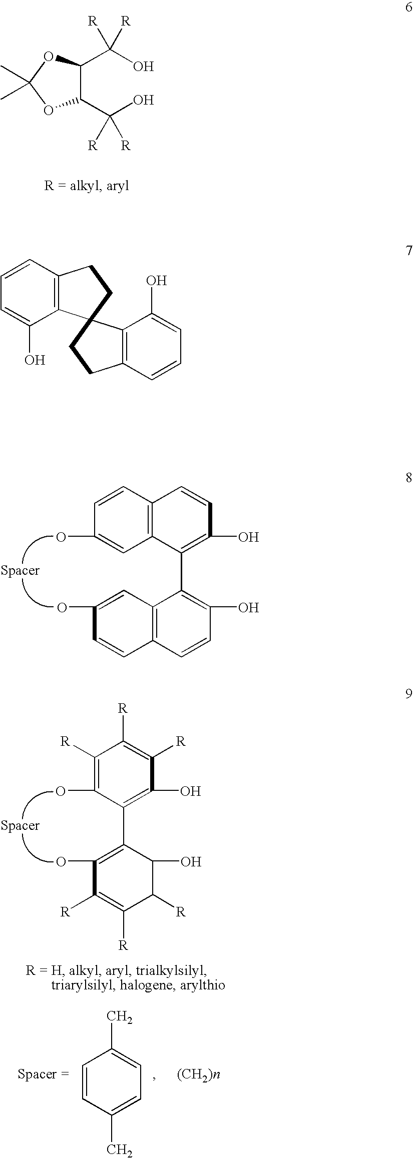
(wherein aryl is an aryl group mentioned above; trialkylsilyl is a trialkylsilyl group such as trimethylsilyl and triethylsilyl; triarylsilyl is a triarylsilyl group such as triphenylsilyl; halogene is a halogen atom mentioned above; arylthio is an arylthio group mentioned above; n is a natural number (preferably, 1 to 10). Hereinafter the same)

Specific examples of the chiral Lewis acid include compounds described in non-patent documents Nos. 3 to 7, compounds described in “Strategy for the design of homogeneous catalysis” (Published by Kagaku-Dojin Publishing Company, INC), p 177-192, compounds described in Yamamoto, H “Lewis Acid in Organic Synthesis”; Wiley-VCH: New York, 2000, and chiral Lewis acids represented by the formulae mentioned below. As such a chiral Lewis acid, commercially available one or appropriately manufactured one may be used.

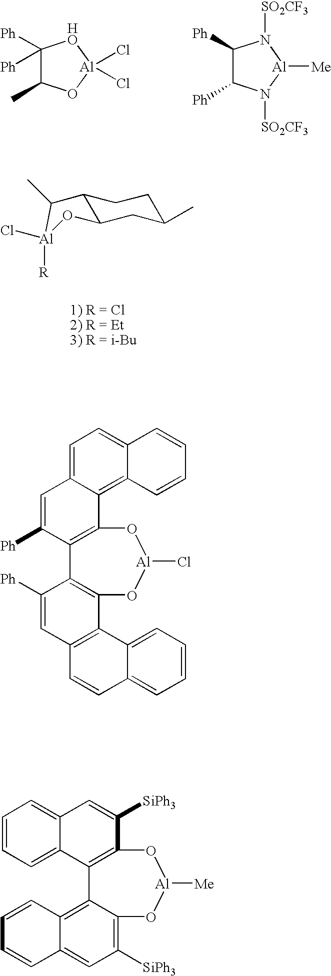

In the above formulae, Ph is phenyl; Me is methyl; Et is ethyl; i-Bu is isobutyl (hereinafter the same). In cases of 1) R═Cl, 2) R=Et or 3) R=i-Bu, R represents chloro, ethyl or isobutyl, respectively.

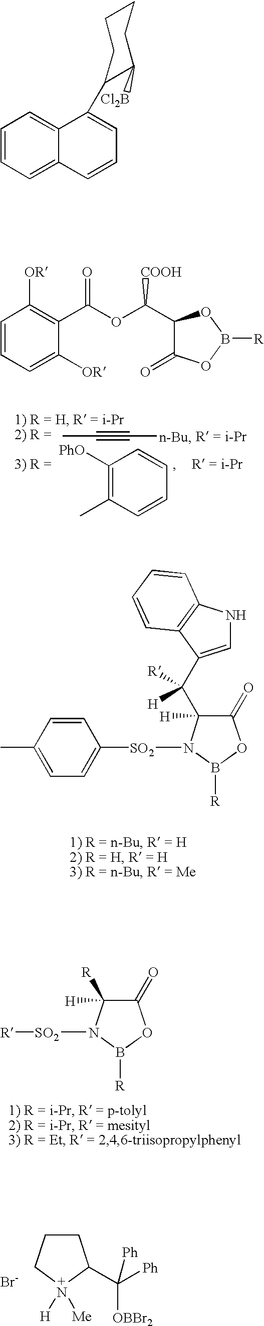

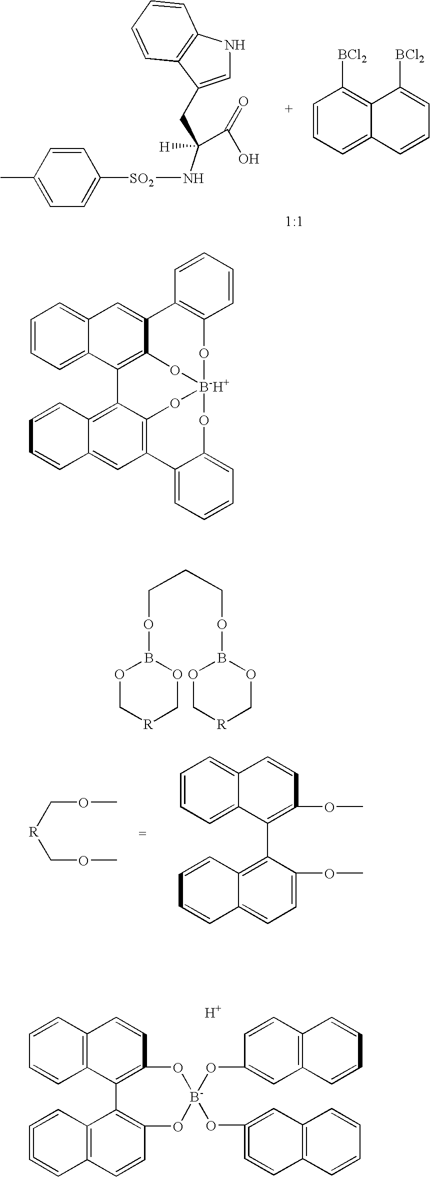

In the above formulae, R or R′ has choices in 1), 2) and 3) in the same way as mentioned above, and i-Pr is isopropyl, n-Bu is n-butyl, p-tolyl is p-tolyl, and mesityl is mesityl (hereinafter the same). 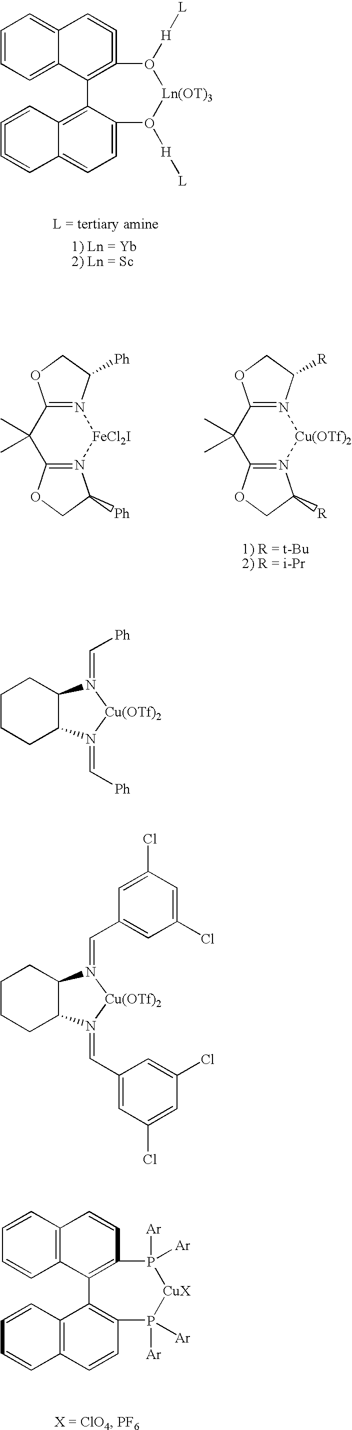
(wherein Ln or R has choices in 1) or 2 ) in the same way as mentioned above, and Ar is an aryl group mentioned above, Tf is trifluoromethanesulfonyl, tertiary amine is, for example, trimethylamine, triethylamine, etc. Hereinafter the same) 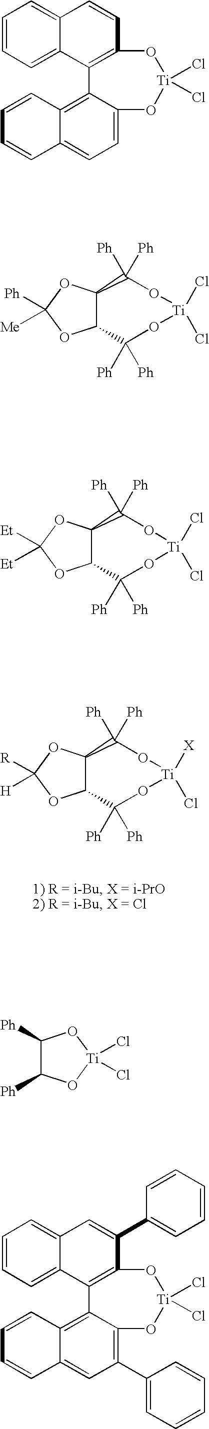 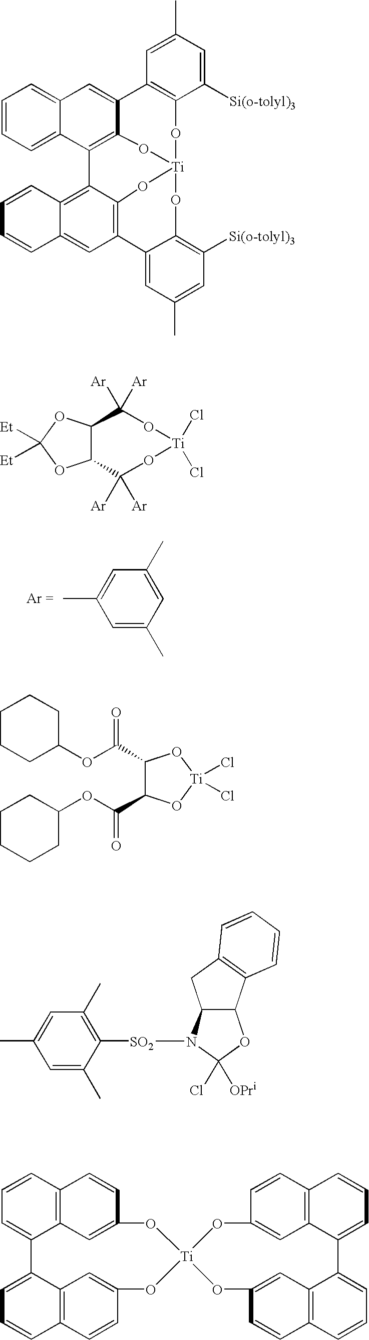
(wherein o-tolyl is an o-tolyl group, OPrⁱ is an isopropoxy group.) 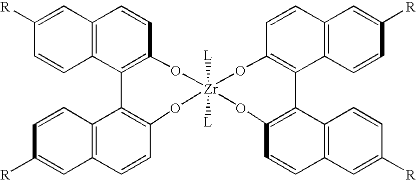
L=tertiary amine
R═H, alkyl, aryl, trialkylsilyl, triarylsilyl, halogene, arylthio 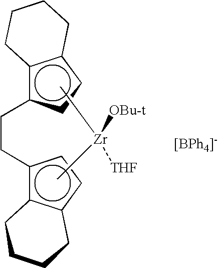
   (wherein OBu-t is t-butoxy, and THF is tetrahydrofuran)

In addition, those chiral Lewis acids which are prepared in situ may also be applied to the production method of the invention.

The optically active amines represented by the above formulae (9) and (9a) which are obtained by the production method of the present invention include optically active tetrahydroquinolines (hereinafter referred to as optically active tetrahydroquinolines) represented by the above formula (1) (hereinafter called optically active tetrahydroquinolines (1)), and specific examples are represented by the following formulae (1a) to (1b): 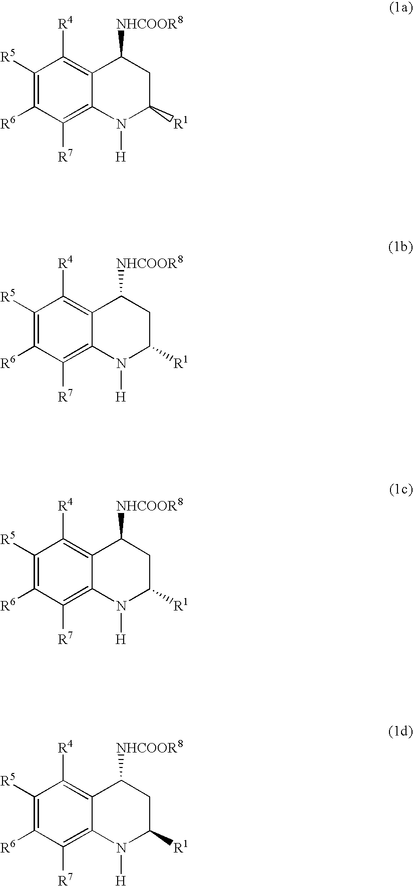
(wherein each symbol has the same meaning as mentioned above)

Among the thus obtained optically active tetrahydroquinolines of formulae (1a) to (1d) above, the tetrahydroquinolines of formulae (1a) and (1b) above are preferable and the tetrahydroquinolines of formulae (1a) above is more preferable. Provided that, when R¹⁰ is a hydrogen atom in the formula (1A), tetrahydoroquinolines of the formula (1A) is tetrahydoroquinolines of the formula (1).

The optically active amines represented by the above formula (9) include, for example, tetrahydroquinolines of formula (1). Examples of the optically active tetrahydroquinolines of formula (1) above (hereinafter called optically active tetrahydroquinolines (1)) include the following compounds:

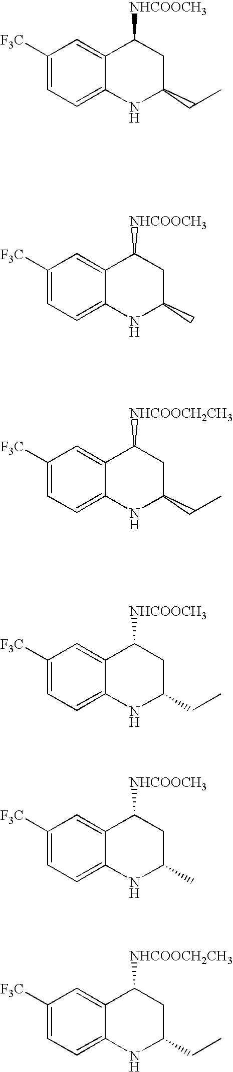

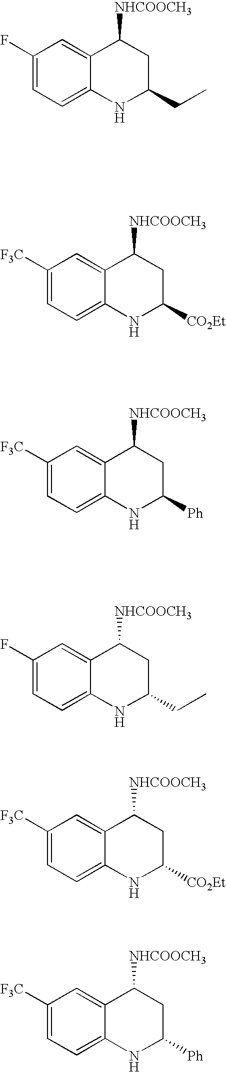

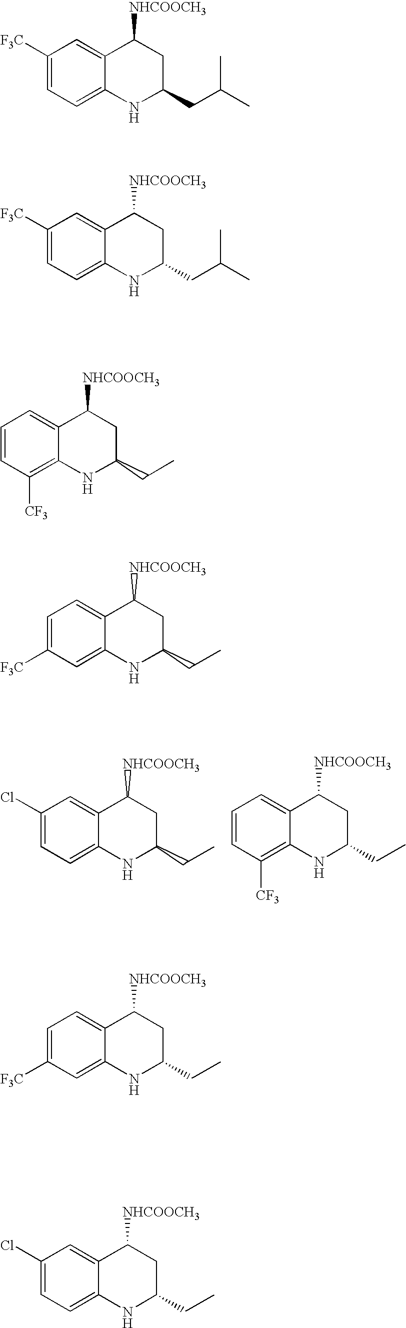

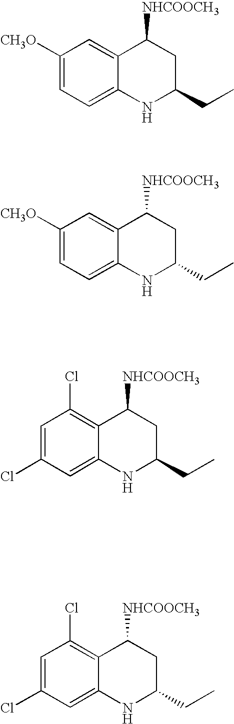

The optically active amines by the formula (10) which are obtained by the production method of the present invention include optically active dihydoroquinolines represented by the formula (1e): 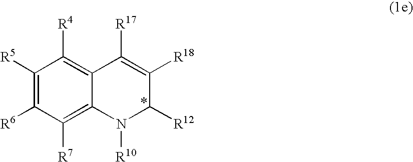
(wherein R⁴ to R⁷, R¹⁰, R¹², R¹⁷, R¹⁸ and the symbol * are each the same as mentioned above), and specific examples are represented by the following compounds (1f) and (1g): 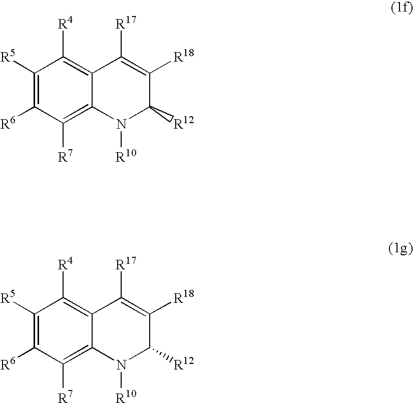
(wherein R⁴ to R⁷, R¹⁰, R¹², R¹⁷ and R¹⁸ are each the same as mentioned above).

Examples of the optically active amines represented by the above formula (10) include the following compounds:

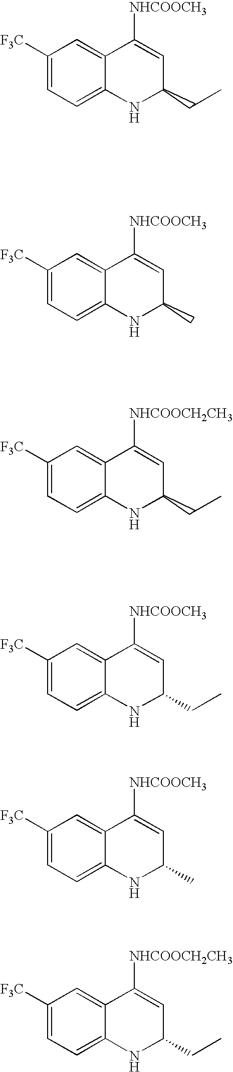

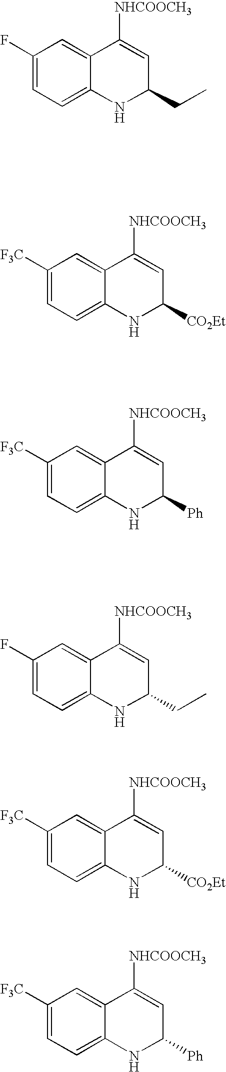

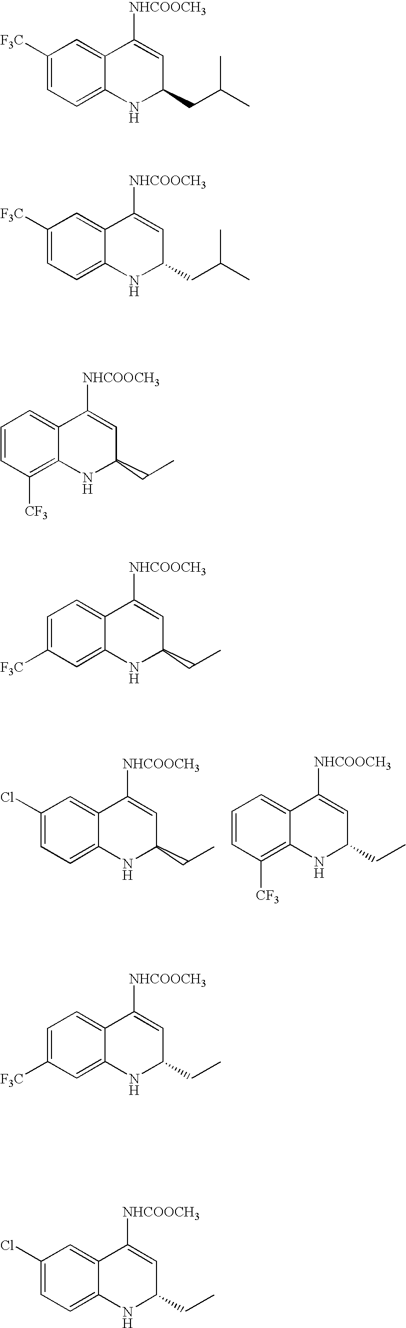

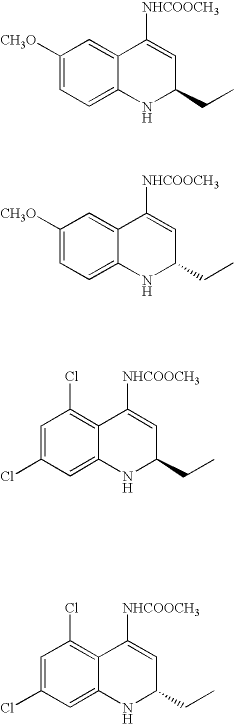

The main reaction participating in the present invention in which an imine equivalent is used as starting compound in the presence of a chiral catalyst is performed according to the following formula:

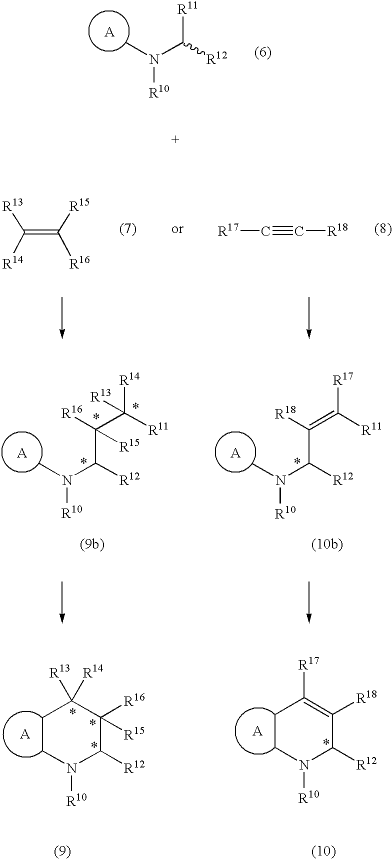

The production method of the present invention is carried out by the reaction of compound (6) with compound (7) or compound (8) in the presence of a chiral catalyst to give optically active compound (9b) or (10b), and furthermore, by cyclization of the optically active compound (9b) or (10b) to give final compound (9) or (10). Therefore, in the production method of the present invention, the reaction may be stopped at the stage when optically active compound (9b) or (10b) is produced, or a mixture of optically active compound (9b) or (10b) and final optically active compound (9) or (10) is produced, or all of the optically active compound (9b) or (10b) are cyclized to give final optically active compound (9) or (10). Thus, the desired compounds in the production method of the present invention in which compound (6), compound (7) or compound (8) and a chiral catalyst as the starting compound are the above optically active compound (9b) or (10b), the mixtures of optically active compound (9b) or (10b) and final compounds or final compounds. The method of producing final compound in accordance with the present invention by use of the above starting compounds has both novelty and technical inventive step. The above optically active compound (9b) or (10b) are new and useful compounds which contribute to said production method of the present invention.

The reaction of imine equivalents (2) or imines (2a) with N-vinyl carbamates (3) in the presence of chiral Lewis acid is explained with an example as the detailed description of the preferred embodiment of the present invention.

The imines (2a) can be obtained by the reaction of amines (4) with aldehydes represented by formula (5) (hereinafter, may be called aldehyde (5)). The amount used of aldehyde (5) is usually selected appropriately from a range of 0.1 to 20 equivalents or preferably 0.3 to 5.0 equivalents to the amines (4).

The reaction is preferably carried out in the presence of a solvent, but it may be carried out without any solvent, depending on the kind of the substrate. The solvents include, for example, aliphatic hydrocarbons such as pentane, hexane, heptane, octane, decane, cyclohexane, etc., aromatic hydrocarbons such as benzene, toluene, xylene, etc., halogenated hydrocarbons such as dichloromethane, 1,2-dichloroethane, chloroform, carbon tetrachloride, o-dichlorobenzene, etc., ethers such as diethyl ether, diisopropyl ether, tert-butyl methyl ether, dimethoxyethane, ethyleneglycol diethyl ether, tetrahydrofuran, 1,4-dioxane, 1,3-dioxolane, cyclopentyl methyl ether, etc., ketones such as acetone, methyl ethyl ketone, methyl isobutyl ketone. Cyclohecanone, etc., alcohols such as methanol, ethanol, 2-propanol, n-butanol, 2-ethoxyethanol, benzyl alcohol, etc., polyalcohols such as ethylene glycol, propylene glycol, 1,2-propanediol, glycerin, etc., esters such as methyl acetate, ethyl acetate, butyl acetate, methyl propionate, etc., amides such as formamide, N,N-dimethylformamide, N, N-dimethylacetamide, etc., sulfoxides such as dimethyl sulfoxide, etc., cyano-containing organic compounds such as acetonitrile, etc., N-methylpyrrolidone, and water. These solvents may be used solely or in appropariate combination of two or more kinds of solvents.

Usually, the amount used of solvents is selected appropriately from a range of 0.1 to 100 times the amount or preferably from a range of 0.5 to 30 times the amount to that of the amines (4).

The reaction temperature is usually selected appropriately from a range of −78 to 100° C. or preferably from a range of −78 to 50° C.

The reaction time is usually selected appropriately from a range of 1 minute to 10 days or preferably from a range of 5 minutes to 48 hours.

The resulting imines are usually subjected to post-treatment or to the subsequent reaction without any post-treatment.

Also, the imine also can be obtained by the appropriate treatment of imine equivalents.

The imine equivalents (2) can be obtained by reacting an amine (4) with an aldehyde (5) and a compound capable of forming an imine equivalent.

The amount of aldehyde (5) used is usually selected appropriately from a range of 0.1 to 20 equivalents or preferably from a range of 0.3 to 5 equivalents to that of the amines (4).

Usually, the amount of a compound capable of forming an imine equivalent is selected appropriately from a range of 0.1 to 20 equivalents or preferably from a range of 0.3 to 5 equivalents to that of amines (4).

The reaction is preferably carried out in the presence of a solvent. The kind and the amount of solvents used are the same as mentioned above.

The reaction temperature is usually selected appropriately from a range of −78 to 200° C. or preferably from a range of −50 to 100° C.

The reaction time is usually selected appropriately from a range of 1 minute to 10 days or preferably from a range of 5 minutes to 48 hours.

The obtained imine equivalent may be subjected to post-treatment or to the subsequent reaction without any post-treatment.

Then the optically active tetrahydroquinolines (1) can be obtained by reacting an imine (2a) or an imine equivalent (2) with a N-vinyl carbamate (3) in the presence of a chiral Lewis acid.

The amount of N-vinyl carbamates (3) used is usually selected appropriately from a range of 0.1 to 50 equivalents or preferably from a range of 0.3 to 10 equivalents to that of imines or imine equivalents.

The chiral Lewis acid is used preferably in a catalytic amount, and when the imine is reacted with the N-vinylcarbamate, the amount used of the chiral Lewis acid is selected appropriately from a range of 0.001 to 10 equivalents, preferably 0.001 to 0.3 equivalents, to that of the imine. Also, in the case that when the imine equivalent is reacted with the N-vinylcarbamate, the amount used of the chiral Lewis acid is selected appropriately from a range of 0.001 to 10 equivalents, preferably 0.001 to 0.3 equivalents, to that of the imine equivalent.

The reaction is carried out preferably in the presence of a solvent. The solvents include, for example, aliphatic hydrocarbons such as pentane, hexane, heptane, octane, decane, cyclohexane, etc., aromatic hydrocarbons such as benzene, toluene, xylene, etc., halogenated hydrocarbons such as dichloromethane, 1,2-dichloroethane, chloroform, carbon tetrachloride, o-dichlorobenzene, etc., ethers such as diethyl ether, diisopropyl ether, tert-butylmethyl ether, dimethoxyethane, ethyleneglycol diethyl ether, tetrahydrofuran, 1,4-dioxane, 1,3-dioxolane, cyclopentyl methyl ether, etc., ketones such as acetone, methyl ethyl ketone, methyl isobutyl ketone. Cyclohexanone, etc., alcohols such as methanol, ethanol, 2-propanol, n-butanol, 2-ethoxyethanol, benzyl alcohol, etc., polyalcohols such as ethylene glycol, propylene glycol, 1,2-propanediol, glycerin, etc., esters such as methyl acetate, ethyl acetate, n-butyl acetate, methyl propionate, etc., amides such as formamide, N, N-dimethylformamide, N,N-dimethylacetamide, etc., sulfoxides such as dimethyl sulfoxide, etc., cyano-containing organic compounds such as acetonitrile, etc., N-methylpyrrolidone, and water. These solvents may be used solely or in combination with two or more kinds of solvents.

Usually, the amount used of solvents is selected appropriately from a range of 0.1 to 100 times the amount or preferably from a range of 0.5 to 30 times the amount to that of the imines or imine equivalents.

The production method of the present invention can be carried out optionally in the presence of inert gas. The inert gas includes nitrogen, argon, etc.

The production method of the present invention may be, as appropriate, carried out optionally in the presence of a dehydration agent. Such dehydration agents include solid oxides such as silica gel, alumina, silica alumina, etc., inorganic dehydration agents such as concentrated sulfuric acid, phosphorous pentoxide, anhydrous zinc chloride, polyphosphoric acid, acid anhydride such as acetic anhydride, carbonyl dimidazole, p-tolenesulfonyl chloride, etc., zeolites such as molecular sieves(3A, 4A or the like), etc., (anhydrous) inorganic salts such as anhydrous calcium chloride, anhydrous calcium sulfate, anhydrous magnesium chloride, anhydrous magnesium sulfate, anhydrous potassium carbonate, anhydrous potassium sulfide, anhydrous potassium sulfite, anhydrous sodium sulfate, anhydrous sodium sulfite, anhydrous copper sulfate, etc., heteropolyacids (more than one water molecule may be added to heteropolyacid, or heteropolyacid may be deposited on a carrier) such as H₃PW₁₂O₄₀, H₃PW₁₁MoO₄₀, H₃PW₁₀Mo₂O₄₀, H₃PW₉Mo₃O₄₀, H₃PW₈Mo₄O₄₀, H₃PVW₁₁O₄₀, H₃PV₂W₁₀O₄₀, H₃PV₃W₉O₄₀, H₃PV₄W₈O₄₀, H₄SiW₁₂O₄₀, H₄SiW₁₀O₄₀, H₄SiW₉Mo₃O₄₀, H₄SiW₈Mo₄O₄₀, H₄SiVW₁₁O₄₀, H₄SiV₂W₉O₄₀, H₄SiV₄W₈O₄₀, etc., cation-exchange resins such as styrene sulfonic acid type, phenol sulfonic acid type, fluorinated alkylsulfonic acid type, etc.

Specific examples of the cation-exchange resin include Amberlyst 15 (registered trademark), Amberlyst 16 (registered trademark) Amberlyst 36 (registered trademark), Amberlite XE-284 (registered trademark) (all these Amberlysts are products of Rohm & Haas), etc., Nafion (registered trademark) (Product of E. I. DuPont), etc. A cation-exchange resin may be deposited on a carrier. The carrier includes silica, etc.

The amount of a dehydration agent is usually selected appropriately from a range of 0.1 to 5.0 equivalents or preferably from a range of 0.5 to 2.0 equivalents to that of the imine equivalent (6) or imine (6a).

When an imine (2a) is reacted with a N-vinyl carbamate in the presence of a chiral Lewis acid, the reaction may be carried out in the coexistence of a compound capable of forming an imine equivalent.

The production method of the invention can be carried out both in a batch process and in a continuous process.

The chiral Lewis acid used in the production method of the invention can be recovered and reused. The recovered chiral Lewis acid may be directly reused without after-treatments and purification for the production method.

In accordance with the present invention, optically active compounds represented by the above formula (9b) or (10b) can be obtained. For example, when imine equivalents represented by the above formula (6) are reacted with alkenes represented by the above formula (7), optically active amines represented by the above formula (9), optically active compounds represented by the above formula (9b) or the mixtures of optically active amines represented by the above formula (9) and optically active compounds represented by the above formula (9b). In the above reaction, when the obtained compound contains substantially only optically active amines represented by the above formula (9), post-treatment may be appropriately carried out. In the case that the obtained compound contains substantially only optically active compounds represented by the above formula (9b), cyclization may be carried out. Also, in the case that the obtained compound contains the mixture of optically active amines represented by the above formula (9) and optically active compounds represented by the above formula (9b), the mixture itself may be cyclized, or optically active compounds represented by the above formula (9b) may be cyclized after isolation of the optically active amines.

Concrete means in the post-treatment or isolation include the known means per se by which optically active amines represented by the above formula (9) and/or optically active compounds represented by the above formula (9b) are separated and purified, and specific examples of such means include, for example, solvent extraction, component transfer, salting out, crystallization, recrystallization, chromatography, etc.

The optically active amines represented by formula (10) and/or optically active compounds represented by formula (10b) may be subjected to cyclization and post-treatment in the similar way as mentioned above.

The optically active compounds represented by the above formula (9b) are preferably those represented by the above formula (9c), more preferably those represented by the following formula (9d): 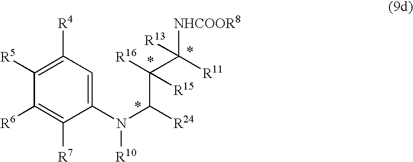
(wherein R⁴ to R⁷, R⁸, R¹⁰, R¹¹, R¹³, R¹⁵, R¹⁶, R²⁴ and the symbol * are each the same as mentioned above)

In the formula (9d), a hydrocarbon group of R²⁴ is preferable, an alkyl group is more preferable, and an ethyl group is furthermore preferable.

Specific examples of the optically active compound represented by the above formula (9d) include, for example, compounds represented by the following formulae:

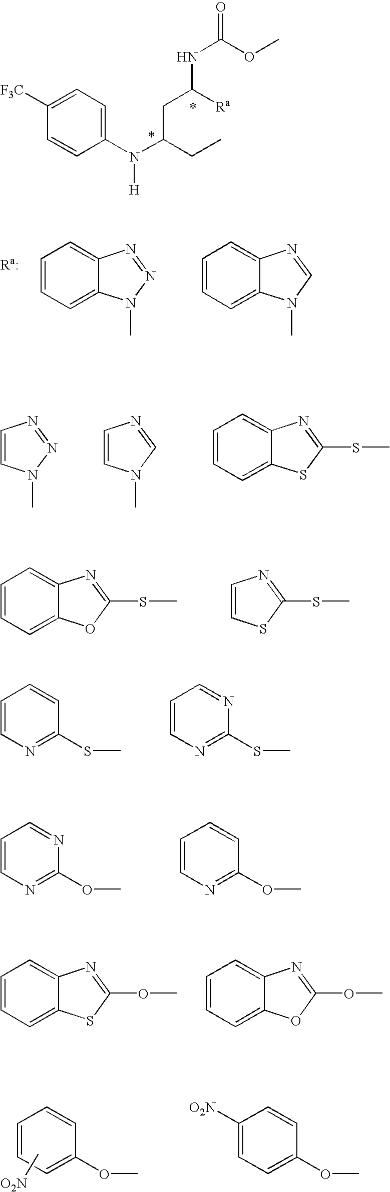

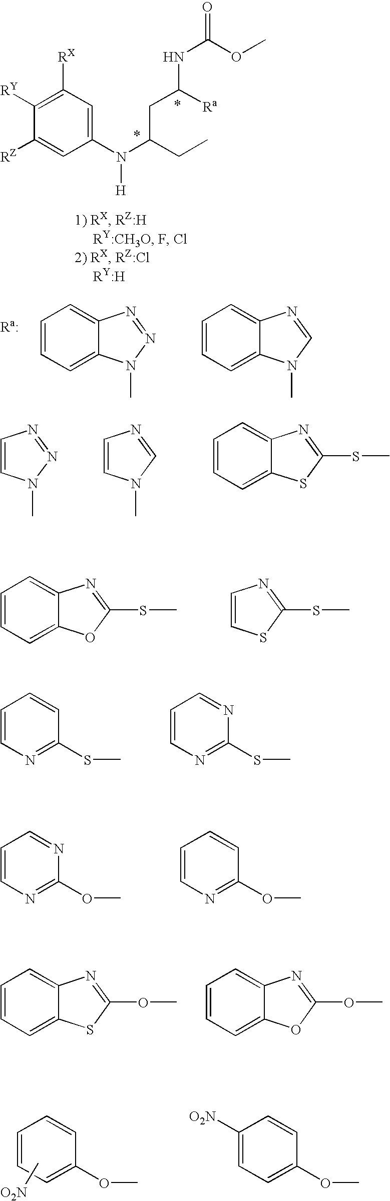

In the above-mentioned formulae, the symbol * is the same as mentioned above.

The optically active compounds represented by the above formula (10b) are preferably those represented by the above formula (10c), more preferably those represented by the following formula (10d): 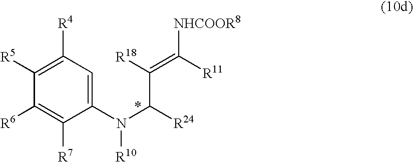
(wherein R⁴ to R⁷, R⁸, R¹⁰, R¹¹, R¹⁸, R²⁴ and the symbol * are each the same as mentioned above).

In the formula (10d), a hydrocarbon group of R²⁴ is preferable, an alkyl group is more preferable, and an ethyl group is furthermore preferable.

Specific examples of the compound represented by the above formula (10d) include, for example, compounds represented by the following formula:

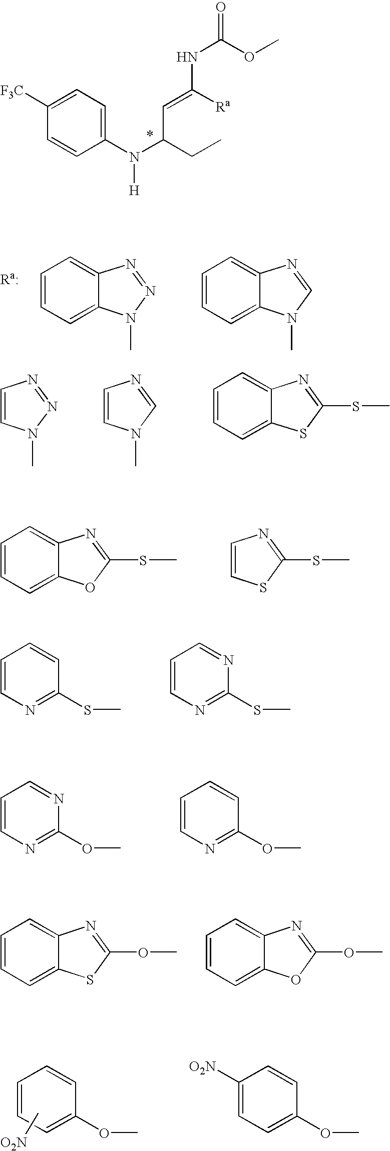

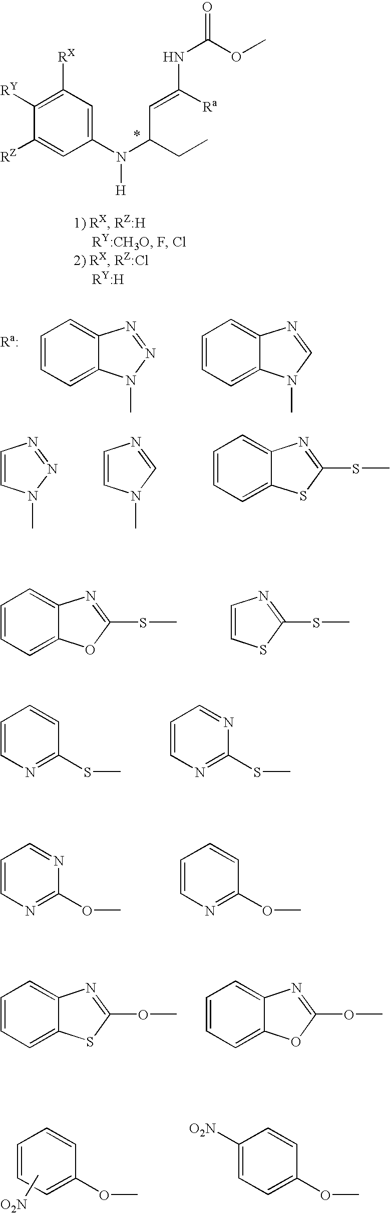

In the above-mentioned formulae, the symbol * is the same as mentioned above.

In the case that the optically active compounds represented by the above formula (9b) wherein R¹¹ is a heterocycle group, the cyclization may be effected while R¹¹ is the heterocycle group or after said heterocycle group is substituted by other leaving group.

The optically active compounds represented by formula (9b) in which a leaving group other than said heterocyclic group is introduced can be obtained, for example, by reacting an optically active compound represented by the above formula (9b) with an alkali metal alkoxide such as sodium methoxide, sodium ethoxide or the like according to the known method. Alternatively, optically active compounds represented by formula (9b) in which a leaving group other than said heterocyclic group is introduced can be obtained in a conventional manner by reacting an optically active compound represented by the above formula (9b) with a nucleophilic agent such as alcohols including methanol and the like exemplified as a solvent mentioned above.

The cyclization may be carried out in one pot after the reaction of imine equivalents represented by the above formula (6) with alkenes represented by the above formula (7), or may be carried out after post-treatment of the reaction, wherein acids and moreover chiral catalysts, dehydration agents or the like may be appropriately added, if necessary.

Such acids include inorganic acids, organic acids, Lewis acids, etc.

Examples of the inorganic acids include, for example, hydrochloric acid, hydrobromic acid, sulfuric acid, phosphoric acid, tetrafluoroboric acid, perchloric acid, periodic acid, etc. Examples of the organic acids include, for example, carboxylic acids such as formic acid, acetic acid, valeric acid, hexanoic acid, citric acid, chloroacetic acid, dichloroacetic acid, trichloroacetic acid, trifluoroacetic acid, benzoic acid, salicylic acid, oxalic acid, succinic acid, malonic acid, phthalic acid, tartaric acid, malic acid, glycolic acid, etc., and sulfonic acids such as methanesulfonic acid, benzenesulfonic acid, p-toluenesulfonic acid, trifluoromethanesulfonic acid, etc. Examples of the Lewsis acids include, for example, aluminum halogenides such as aluminum chloride, aluminum bromide, etc., dialkylaluminum halogenides such as diethylaluminum chloride, diethylaluminum bromide, diisopropylaluminum chloride, etc., trialkyl borates such as trimethyl borate, triethyl borate, tripropyl borate, tri-tert-butyl borate, etc., trialkoxyaluminums such as triethoxyaluminum, triisopropoxyaluminum, tri-tert-butoxyaluminum, etc., titanium halogenides such as titanium tetrachloride, etc., tetraalkoxytitanium such as tetraisopropoxytitanium, etc., boron halogenides such as boron trifluoride, boron trichloride, boron tribromide, boron trifluoride diethyl etherate, etc., zinc halogenides such as zinc chloride, zinc bromide, etc. These acids may be used solely or in combination of two or more kinds of acids. Among these acids, sulfuric acid, hydrochloric acid, methanesulfonic acid, and trifluoromethanesulfonic acid are preferable.

The amount used of the acid is usually selected appropriately from a range of 0.001 to 10 equivalents or preferably 0.001 to 0.3 equivalents to that of the imine equivalent (6) or the imine (6a). The reaction temperature is usually selected appropriately from a range of −78 to 200° C. or preferably −50 to 100° C. The reaction time is usually selected appropriately from a range of 1 minute to 10 days, preferably 5 minutes to 48 hours.

The optical purity of the optically active tetrahydroquinolines (1) obtained by the production method of the present invention preferably equal to or higher than 85% e.e., more preferably 90% e.e.

Thus obtained optically active amines are optically active tetrahydroquinolines such as 1,2,3,4-tetrahydroquinolines or optically active dihydroquinolines such as 1,2-dihydroquinolines, preferably optically active 1,2,3,4-tetrahydroquinolines.

Further, the production method of the present invention may include, as a whole, cases other than said preferred embodiments, and other cases encompassed in the present invention other than said preferred embodiments are carried out in the same way as in said preferred embodiments.

In addition, in the description as mentioned above, the carbon atom at the position represented by the symbol * is an asymmetric carbon atom (see, for example, formulae (9), (9a-1), (9b), (9c), (10), (10a), (10b) and (10c)). However, in the case that two groups which are bound to the carbon atom at the position of the symbol * are each the same, said carbon atom is, as a matter of course, not asymmetric carbon atom. For example, carbon atom to which R¹⁵ and R⁶ bind in the case that the groups represented by R¹⁵ and R¹⁶ are each the same are not an asymmetric carbon. Consequently, carbon atoms to which R¹² or R¹⁵ and R¹⁶ bind may be generally asymmetric or not asymmetric. The present invention includes both of those cases. However, since R¹³ and R¹⁴ cannot be each the same, the carbon atoms to which R¹³ and R¹⁴ bind are an asymmetric carbon atom at all times. Therefore, the compounds obtained by the production method of the present invention may contain always at least one asymmetric carbon atom. However, R¹² is, among the groups as defined above, preferably a group other than a hydrogen atom.

### EXAMPLES

The present invention is illustrated in more detail by referring to the following Examples and Reference Examples. However, the present invention is not restricted in its scope by these Examples.

Apparatuses used in the following Examples and Reference Examples for measuring physical constants are as follows: Nuclear Magnetic Resonance:

(1) DRX500 (BRUKER JAPAN CO.LTD.) ¹H-NMR (500.13 MHz).

(2) Gemini 2000 (Varian) ¹H-NMR (200 MHz)

Melting Point: Yanaco MP-500D

High Performance Liquid Chromatography (HPLC): Shimadzu Seisakusho LC10AT & SPD10A

Mass Spectrum (MASS): Hitachi M-80B

### Reference Example 1

Preparation of chiral Lewis acid represented by the following formula:

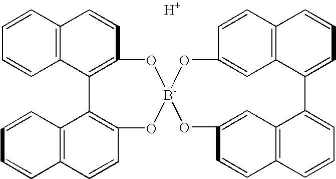

Under nitrogen atmosphere, a mixture of (R)-binaphthol 50 mg (0.17 mmol), 1M-trimethoxyboran-dichloromethane 0.09 mL (0.09 mmol) and dichloromethane 10 mL was refluxed for 3 hours with a cooling tube loaded with molecular sieve 4A 4 g to give a dichloromethane solution (0.09 mmol) of chiral Lewis acid represented by the formula mentioned above. The solution was then concentrated under reduced pressure to a total volume of 5.0 ml to give 0.018M (chiral Lewis acid)-dichloromethane solution.

### Example 1

### Synthesis of methyl (2R,4S)-(2-ethyl-6-trifluoromethyl-1,2,3,4-tetrahydroquinolin-4-yl)-carbamate

4-Trifluoromethylaniline 161.3 mg (1.0 mml) and propionaldehyde 58.1 mg (1.0 mmol) in dichloromethane 5.0 mL were mixed and stirred at ambient temperature for 3 hours. Then, the solution of N-propylidene-4-trifluoromethylphenylamine in dichloromethane 2.5 mL obtained above, methyl N-vinylcarbamate 111.0 mg (1.1 mmol) and the solution 2.5 mL (0.045 mmol) of 0.018 M (chiral Lewis acid)-dichloromethane prepared in Reference Example 1 were mixed and stirred at −30° C. for 5 hours. Upon completion of the reaction, 2.5 wt % aqueous sodium bicarbonate 2.0 mL was added to the reaction mixture and the mixture was stirred for 10 minutes. The organic layer was washed with water 3.0 mL and dried over anhydrous magnesium sulfate. After removal of the solvent by evaporation in vacuo, the residue was purified by silica gel column chromatography (n-hexane:ethyl acetate=3:1) to give the title compound 67.9 mg in 22.5% yield.

Optical purity: 91.3% ee. GC-MS: 302(M⁺)

¹H-NMR (500 MHz, CDCl₃):1.00 (t, 3H, J=7, 4 Hz), 1.51-1.62 (m, 3H),

2.19 (ddd, 1H, J=2, 8 Hz, 5.5 Hz, 12.2 Hz), 3.46-3.52 (m, 1H), 3.67 (s, 3H), 4.90-4.95 (m, 1H), 5.62 (brs, 1H), 6.51 (brd, 1H, J=8.7), 6.65 (d, 1H, J=8.7), 7.20 (d, 1H, J=8.7 Hz), 7.31 (s, 1H).

### Example 2

### Synthesis of methyl (2R,4S)-(2-ethyl-6-trifluoromethyl-1,2,3,4-tetrahydro quinolin-4-yl)-carbamate

### (1) Synthesis of (1-benzotriazole-1-ylpropyl)-(4-trifluoromethylphenyl)amine

Under nitrogen atmosphere, 4-trifluoromethylaniline 30.0 g (186.0 mmol) and propionaldehyde 11.88 g (204.6 mmol) were added successively to a mixture of benzotriazole 22.16 g (186.0 mmol) and toluene 90 mL at ambient temperature, and the mixture was stirred at ambient temperature for 18 hours. Upon completion of the reaction, n-heptane 100 ml was added to the reaction mixture. Then, the mixture was cooled gradually to −10° C, and stirred for 3 hours. The precipitated crystals were filtered to give the desired (1-benzotriazol-1-ylpropyl)-(4-trifluoromethylphenyl)amine 54.0 g in 90.6% yield.

¹H-NMR (200 MHz, CDCl₃):0.97 (t, 3H, J=7, 4 Hz), 2.37 (m, 2H), 5.04 (brd, 1H, J=7.8), 6.31 (m, 1H), 6.74 (d, 2H, J=8.4 Hz), 7.30-7.46 (m, 4H), 7.69 (d, 1H, J=8.0 Hz), 8.09 (d, 1H, J=8.4 Hz)

### (2) Synthesis of methyl (2R,4S)-(2-ethyl-6-trifluoromethyl-1,2,3,4-tetrahydroquinolin-4-yl)-carbamate

Under nitrogen atmosphere, a solution of methyl N-vinyl carbamate 202.2 mg (2.0 mmol) and dichloromethane 5.0 mL was added to a dichloromethane solution 3.0 mL (0.138 mmol) of 0.046 M-(chiral Lewis acid) prepared by the method similar to Reference Example 1, in an ice bath. Then, to the mixture in an ice bath was added dropwise a solution of (1-benzotriazol-1-ylpropyl)-(4-trifluoromethylphenyl)amine 320.3 mg (1.0 mmol) in dichloromethane 5.0 mL and the mixture was stirred for 4 hours. After completion of the reaction, to the reaction mixture were added 2.5 wt % sodium bicarbonate 2.0 mL and ethyl acetate 30.0 mL, and then the mixture was stirred for 10 minutes. The organic layer was washed with water 3.0 mL, dried over anhydrous magnesium sulfate and the residue was purified by silica gel column chromatography (n-hexane:ethyl acetate=3:1) to give the desired aminoquinoline 202.7 mg in 67.1% yield. Optical purity: 97.9% ee. The spectrum of ¹H-NMR was identical to that of Example 1.

### Reference Example 2

Preparation of chiral Lewis acid represented by the following formula:

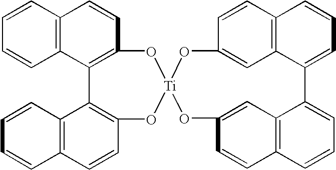

Under nitrogen atmosphere, (R)-binaphthol 50 mg (0.17 mmol) was suspended in toluene (1 mL), and titanium tetraisopropoxide 26 μL (0.09 mmol) was added dropwise to give a homogeneous brown solution. The solution was stirred at ambient temperature for 30 minutes to give the desired chiral Lewis acid in toluene.

### Example 3

### Synthesis of methyl (2R, 4S)-(2-ethyl-6-trifluoromethyl-1,2,3,4-tetrahydroquinolin-4-yl)-carbamate

To a solution of chiral Lewis acid in toluene solution, obtained in Reference Example 2, was added 1.0 mL of dichloromethane, and then, to the mixture was added solution of (1-benzotriazol-1-ylpropyl)-(4-trifluoromethylphenyl)amine 320.3 mg (1.0 mmol) and methyl N-vinylcarbamate 111.0 mg (1.0 mmol) in toluene (2.2 mL) and dichloromethane (2.2 mL). The reaction mixture was stirred at 20° C. for 2 hours. Upon completion of the reaction, to the reaction mixture was added 2.5 wt % aqueous sodium bicarbonate 2.0 mL, and the reaction mixture was stirred for 10 minutes. The organic layer was washed with water 3.0 mL and dried over anhydrous magnesium sulfate. After removal of the solvent by evaporation in vacuo, the residue was purified by column chromatography on silica gel with n-hexane:ethyl acetate=3:1 to give the desired aminoquinoline 115 mg in 38% yield. Optical purity: 50% ee. The spectrum of ¹H-NMR was identical to that of Example 1.

### Reference Example 3

Preparation of chiral Lewis acid represented by the following formula:

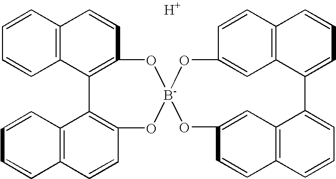

Under nitrogen atmosphere, a mixture of (R)-binaphtol 880 mg (3.07 mmol) and trimethoxyborane 159.7 mg (1.53 mmol) in 26 mL of toluene was stirred at 45 to 50° C. for 2 hours. The reaction mixture was concentrated to give crystals, which were then dissolved in dichloromethane 10 mL to afford the desired solution (0.15 M) of chiral Lewis acid as dichloromethane solution, represented by the above formula.

### Example 4

### Synthesis of methyl (2R,4S)-(2-ethyl-6-trifluoromethyl-1,2,3,4-tetrahydroquinoline-4-yl)-carbamate and (3R)-[1-(benzotriazol-1-yl)-3-(4-trifluoromethylphenylamino)pentyl]-carbamate

Under nitrogen atmosphere, a mixture of (1-benzotriazol-1-ylpropyl)-(4-trifluolomethylphenyl)amine 3.20 g (10.0 mmol) obtained by the method similar to that of Example 2(1) and methyl N-vinylcarbamate 1.21 g (12.0 mmol) in 9.6 mL of dichloromethane was cooled to −20° C. To the mixture was added dropwise the solution of 0.15 M chiral Lewis acid prepared in Reference Example 3, in dichloromethane 3.3 mL (0.5 mmol), and the mixture was stirred at the same temperature for 2 hours. (The analysis of the reaction mixture by high performance liquid chromatography revealed that production ratio of methyl (2R,4S)-(2-ethyl-6-trifluoromethyl-1,2,3,4-tetrahydroquinolin-4-yl)-1-carbamate and methyl (3R)-[1-(benzotriazol-1-yl)-3-(4-trifluoromethylphenyl-amino)pentyl]-carbamate was 16.1:83.9 in conversion rate 99%) Upon completion of the reaction, to the reaction mixture was added toluene 9.6 mL, and the mixture was cooled to −30° C. and stirred for 2 hours. The precipitated crystals were filtered to give the title compound (as a mixture) 0.89 g in 21.1% yield. MS and ¹H-NMR MS: 444 (M+Na⁺) of methyl (3R)-[1-(benzotriazol-1-yl)-3-(4-trifluoromethylphenyl-amino)pentyl]-carbamate

¹H-NMR (500 MHz, CD₃COCD₃):0.98 (t, 3H, J=7, 2 Hz), 1.63-1.74 (m, 2H), 2.40 (m, 1H), 2.88 (m, 1H), 3.49 (s, 3H), 3.74 (m, 1H), 5.02 (brd, 1H, J=9, 4 Hz), 6.70-6.75 (m, 1H), 6.73 (d, 2H, J=8, 4 Hz), 7.36 (d, 2H, J=8, 4 Hz), 7.40 (dd, 1H, J=8, 2 Hz, 6.0 Hz), 7.53 (dd, 1H, J=7, 3 Hz, 6.1 Hz), 7.84-7.93 (m, 2H), 8.01 (d, 1H, J=8, 3 Hz).

### Reference Example 4

Preparation of chiral Lewis acid represented by the following formula:

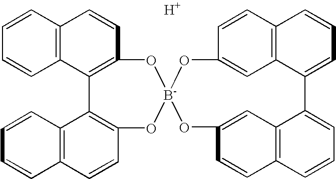

Under nitrogen atmosphere, a mixture of (R)-binaphtol 880 mg (3.07 mmol) and trimethoxyborane 159.7 mg (1.53 mmol) in 26 mL of toluene was stirred at 45 to 50° C. for 2 hours. The reaction mixture was concentrated to a total volume of 10 mL, and cyclopentyl methyl ether 6.0 mL was added to the reaction mixture to give a solution of 0.1 M chiral Lewis acid represented by the above formula, in toluene-cyclopentyl methyl ether.

### Example 5

### Synthesis of methyl (2R,4S)-(2-ethyl-6-trifluoromethyl-1,2,3,4-tetrahydroquinolin-4-yl)-carbamate(including the cyclization process of intermediate)

Under nitrogen atmosphere, a mixture of (1-benzotriazol-1-ylpropyl)-(4-trifluolomethylphenyl)amine 3.20 g (10.0 mmol) prepared by the method similar to that of Example 2(1) and methyl N-vinylcarbamate 1.21 g (12.0 mmol) in 10.0 mL of cyclopentyl methyl ether was cooled to −15° C. To the mixture was added dropwise the solution 5.3 mL (0.5 mmol) of 0.1 M chiral Lewis acid in toluene-cyclopentyl methyl ether prepared in Reference Example 4, and then the mixture was stirred for 2 hours. (The analysis of the reaction mixture by high performance liquid chromatography revealed that production ratio of methyl (2R,4S)-(2-ethyl-6-trifluoromethyl-1,2,3,4-tetrahydroquinolin-4-yl)-carbamate and methyl (3R)-[1-(benzotriazaol-1-yl)-3-(4-trifluoromethylphenylamino)pentyl]-carbamate was 22.5:77.5 in conversion rate 99%). After addition of 28 wt % sodium methoxide 2.32 g (12.0 mmol) in methanol, the reaction mixture was stirred for 3 hours while warming up gradually to room temperature. To the reaction mixture was added 10.0 mL of water, and the reaction mixture was stirred for 15 minutes. The organic layer was washed twice with water 10.0 mL, dried over sodium sulfate and the solvent was evaporated in vacuo to give a residue, which was then dissolved in 10.0 mL of toluene. After addition of p-toluenesulfonic acid monohydrate 38.0 mg (0.2 mmol), the mixture was stirred at 50° C. for 3 hours. Upon completion of the reaction, the reaction mixture was washed with 5 wt % aqueous sodium hydrogen carbonate 10.0 mL and then washed twice with water 10.0 mL. The organic layer was dried over sodium sulfate, and the solvent was evaporated in vacuo to give a residue, which was purified by silica gel column chromatography (n-hexane:ethyl acetate=3:1) to give the title compound 2.75 g in 91.0% yield. Optical purity:93.8% ee.

### Example 6

### Synthesis of methyl (2R,4S)-(2-ethyl-6-trifluoromethyl-1,2,3,4-tetrahydroquinolin-4-yl)-carbamate (Cyclization of metyl (3R)-[1-(benzotriazaol-1-yl)-3-(4-trifluoromethyl-phenylamino)pentyl]-carbamate)

Under nitrogen atmosphere, a solution of 421.4 mg (1.0 mmol) of methyl (3R)-[1-(benzotriazaol-1-yl)-3-(4-trifluoromethyl-phenylamino)pentyl]-carbamate obtained in Example 4 and 19 mg (0.1 mmol) of p-toluenesulfonic acid monohydrate in 2.0 mL of methanol was stirred at 50° C. for 1 hour. To the resulting mixture was added 20 mL of ethyl acetate and 6.0 mL of 0.5 wt % aqueous sodium bicarbonate and stirred 10 minutes. The organic layer was separated, washed twice with 10 mL of water and dried over anhydrous magnesium sulfate. After removal of the solvent by evaporation in vacuo, the product was purified by flash silica gel column chromatography (n-hexane:ethyl acetate=4:1) to give the title-compound 257 mg in 85.0% yield. Optical purity: 99.7%ee.

### INDUSTRIAL APPLICABILITY

Optically active tetrahydroquinolines and dihydroquinolines useful as synthetic intermediates for pharmaceuticals, agrochemicals, etc., can be prepared in accordance with the present invention.

1. A method for producing optically active amines of formula (9): 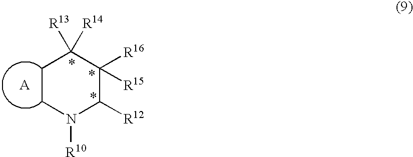
(wherein the group represented by ring A is aryl, substituted aryl, aromatic heterocyclic group or substituted aromatic heterocyclic group; R¹⁰ is hydrogen, alkyl, substituted alkyl, aryl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹² is hydrocarbon, substituted hydrocarbon, COOR¹⁹, (R¹⁹ is a hydrocarbon group or a substituted hydrocarbon group), COR²⁰ (R²⁰ is a substituted amino group) or substituted amino; R¹³, R¹⁵ and R¹⁶ are each independently hydrogen or alkyl; R¹⁴ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, heteroaryloxy, substituted heteroaryloxy, alkylthio, substituted alkylthio, arylthio, substituted arylthio, aralkylthio, substituted aralkylthio, heteroarylthio, substituted heteroarylthio, substituted amino, substituted silyl, alkylseleno, aralkylseleno, arylseleno or heteroarylseleno; R¹⁴ and R¹⁶ taken together may form a ring; the symbol * is an asymmetric carbon atom; 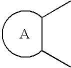
is a divalent group corresponding to the group represented by ring A as mentioned above), or (10): 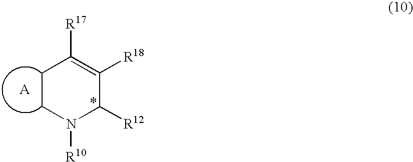
(wherein R¹⁷ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, substituted amino or substituted silyl; R¹⁸ is hydrogen or alkyl; R¹⁰ and R¹² are each the same as mentioned above; the symbol * is an asymmetric carbon atom; and 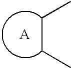
is the same as mentioned above), which comprises reacting an imine equivalent of formula (6): 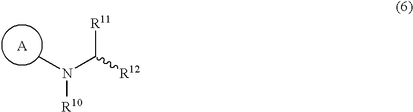
(wherein R¹¹ is a leaving group; and ring A, R¹⁰ and R¹² are each the same meaning as mentioned above) with an alkene of formula (7): 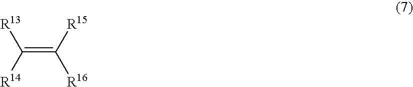
(wherein R¹³, R¹⁴, R¹⁵ and R¹⁶ are each the same as mentioned above) or an alkyne of formula (8):
R¹⁷—C≡C—R¹⁸ (8)
(wherein R¹⁷ and R¹⁸ are each the same as mentioned above) in the presence of a chiral catalyst.

2. The method as claimed in claim 1, wherein an optically active compound of formula (9b): 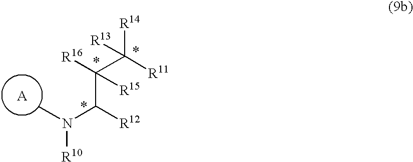
(wherein the symbol * is an asymmetric carbon atom ; the group represented by ring A, and R¹⁰ to R¹⁶ are each the same as mentioned above) or an optically active compound of formula (10b): 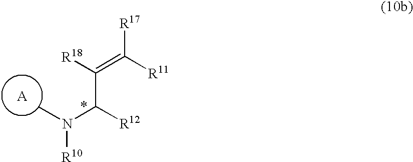
(wherein the symbol * is an asymmetric carbon atom; the group represented by ring A, R¹⁰, R¹¹, R¹², R¹⁷ and R¹⁸ are each the same as mentioned above) is formed in the reaction system.

3. A method for producing optically active amines of formula (9): 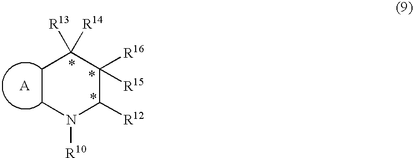
(wherein the group of ring A is aryl, substituted aryl, aromatic heterocyclic group or substituted aromatic heterocyclic group; R¹⁰ is hydrogen, alkyl, substituted alkyl, aryl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹² is hydrocarbon, substituted hydrocarbon, COOR¹⁹ (R¹⁹ is a hydrocarbon group or substituted hydrocarbon group), COR²⁰ (R²⁰ is a substituted amino group), or substituted amino; R¹³, R¹⁵ and R¹⁶ are each independently hydrogen or alkyl; R¹⁴ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, heteroaryloxy, substituted heteroaryloxy, alkylthio, substituted alkylthio, arylthio, substituted arylthio, aralkylthio, substituted aralkylthio, heteroarylthio, substituted heteroarylthio, substituted amino, substituted silyl, alkylseleno, aralkylseleno, arylseleno or heteroarylseleno; and R¹⁴ and R¹⁶ taken together may form a ring; 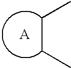
is a divalent group corresponding to the group represented by ring A as mentioned above; and the symbol * is an asymmetric carbon atom) or (10): 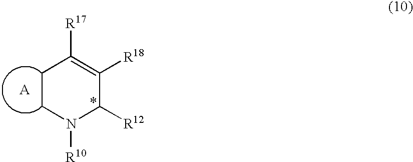
(wherein R¹⁷ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, substituted amino or substituted silyl; R¹⁸ is hydrogen or alkyl; R¹⁰, R¹², ring A, the symbol * and 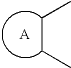
are each the same as mentioned above), which comprises cyclization of an optically active compound of formula (9b): 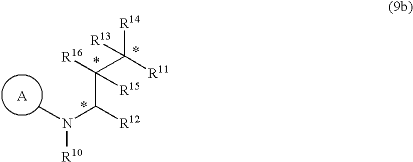
(wherein R¹¹ is a leaving group; the symbol * is an asymmetric carbon atom; the group of ring A and R¹⁰ to R¹⁶ are each the same as mentioned above) or an optically active compound of formula (10b): 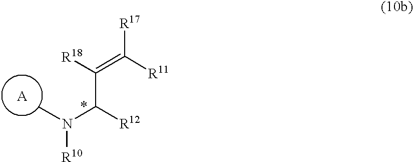
(wherein the group represented by ring A, R¹⁰, R¹¹, R¹², R¹⁷, R¹⁸ and the symbol * are each the same as mentioned above).

4. A method for producing optically active amines of formula (9a-1): 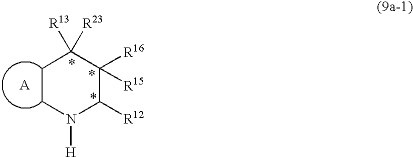
(wherein the group represented by ring A is aryl, substituted aryl, aromatic heterocyclic group or substituted aromatic heterocyclic group; R¹² is hydrocarbon, substituted hydrocarbon, COOR¹⁹ (R¹⁹ is a hydrocarbon group or a substituted hydrocarbon group), COR²⁰ (R²⁰ is a substituted amino group) or substituted amino; R¹³, R¹⁵ and R¹⁶ are each independently hydrogen or alkyl; R²³ is aryl, substituted aryl, aliphatic heterocyclic group, substituted aliphatic heterocyclic group, aromatic heterocyclic group, substituted aromatic heterocyclic group, aryloxy, substituted aryloxy, aralkyloxy, substituted aralkyloxy, heteroaryloxy, substituted heteroaryloxy, alkylthio, substituted alkylthio, arylthio, substituted arylthio, aralkylthio, substituted aralkylthio, heteroarylthio, substituted heteroarylthio, substituted amino, substituted silyl, alkylseleno, aralkylseleno, arylseleno or heteroarylseleno; and R²³ and R¹⁶, taken together may form a ring; 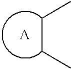
is a divalent group corresponding to the group represented by ring A as mentioned above; and the symbol * is an asymmetric carbon atom) or (10a): 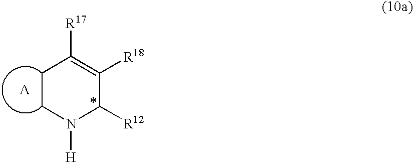
(wherein R¹⁷ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, substituted amino or substituted silyl; R¹⁸ is hydrogen or alkyl; R¹² and 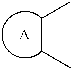
are each the same as mentioned above; and the symbol * is an asymmetric carbon atom), which comprises reacting an imine of formula (6a): 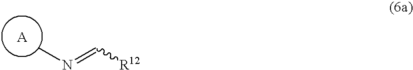
(wherein the group represented by ring A and R¹² are each the same as mentioned above) with an alkene of formula (7a): 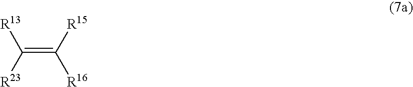
(wherein R¹³, R¹⁵, R¹⁶ and R²³ are each the same as mentioned above), or with an alkyne of formula (8):
R¹⁷—C≡C—R¹⁸ (8)
(wherein R¹⁷ and R¹⁸ are each the same as mentioned above) in the presence of a chiral catalyst.

5. A method for producing optically active 1,2,3,4-tetrahydroquinolines of formula (1A): 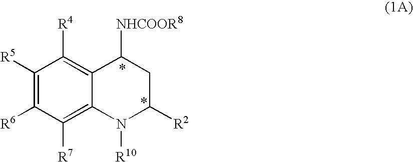
(wherein R² is a hydrocarbon group, a substituted hydrocarbon group or COOR⁹(R⁹ is a hydrocarbon group); R⁴ to R⁷ are each independently hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R⁸ is a hydrocarbon group; R¹⁰ is hydrogen, alkyl, aryl, substituted alkyl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; and R⁴ and R⁵, R⁵ and R⁶, or R⁶ and R⁷, taken together, may form a fused ring, and the symbol * is an asymmetric carbon atom), which comprises reacting an imine equivalent of formula (2): 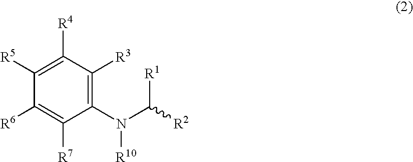
(wherein R¹ is a leaving group; R³ is hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R³ and R⁴, R⁴ and R⁵, R⁵ and R⁶, or R⁶ and R⁷, taken together, may form a fused ring, provided that either of R³ or R⁷ is hydrogen; R² to R⁷ and R¹⁰ are each the same as mentioned above) with a N-vinyl carbamate of formula (3): 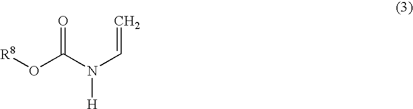
(wherein R⁸ is the same as mentioned above) in the presence of a chiral Lewis acid.

6. A method for producing optically active quinolines of formula (1): 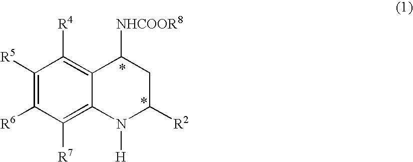
(wherein R² is a hydrocarbon group, a substituted hydrocarbon group or COOR⁹ (R⁹ is hydrocarbon); R⁴ to R⁷ are each independently hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R⁴ and R⁵, R⁵ and R⁶, or R⁶ and R⁷, taken together, may form a fused ring; R⁸ is a hydrocarbon group; and the symbol * is an asymmetric carbon atom), which comprises reacting an imine of formula (2a): 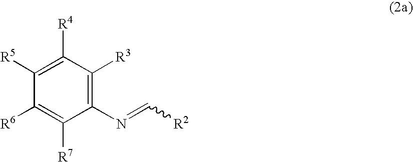
(wherein R³ is hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R³ and R⁴, R⁴ and R⁵, R⁵ and R⁶, or R⁶ and R⁷, taken together, may form a fused ring, provided that either of R³ or R⁷ is hydrogen; R², and R⁴ to R⁷ are each the same as mentioned above) with an N-vinyl carbamate of formula (3): 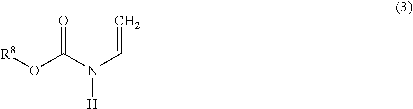
(wherein R⁸ is the same as mentioned above) in the presence of a chiral Lewis acid.

7. A method for producing optically active 1,2,3,4-tetrahydroquinolines of formula (1): 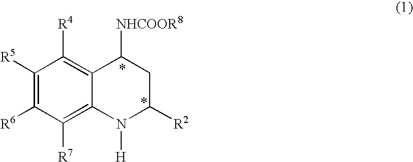
(wherein R² is a hydrocarbon group, a substituted hydrocarbon group or COOR⁹(R⁹ is a hydrocarbon group); R⁴ to R⁷ are each independently hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R⁴ and R⁵, R⁵ and R⁶, or R⁶ and R⁷, taken together, may form a fused ring; R⁸ is a hydrocarbon group; and the symbol * is an asymmetric carbon atom), which comprises reacting an amine of formula (4): 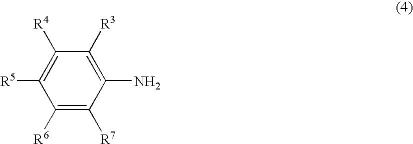
(wherein R³ is hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R³ and R⁴, R⁴ and R⁵, R⁵ and R⁶, or R⁶ and R⁷, taken together, may form a fused ring, provided that either of R³ or R⁷ is hydrogen; R⁴ to R⁷ are each the same as mentioned above), with an aldehyde of formula (5):
R²—CHO (5)
(wherein R² is the same as mentioned above) and a compound capable of forming imine equivalents, and reacting the resulting imine equivalent of formula (2): 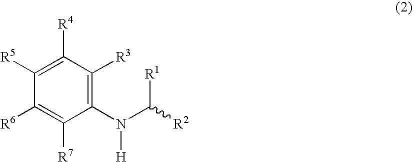
(wherein R¹ is a leaving group; R² to R⁷ are each the same as mentioned above) with an N-vinyl carbamate of formula (3): 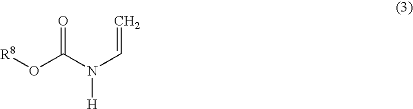
(wherein R⁸ is the same as mentioned above) in the presence of a chiral Lewis acid.

8. A method for producing optically active 1,2,3,4-tetrahydroquinolines of formula (1): 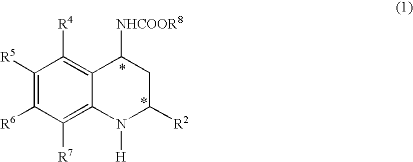
(wherein R² is a hydrocarbon group, a substituted hydrocarbon group or COOR⁹ (R⁹ is a hydrocarbon group); R⁴ to R⁷ are each independently hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R⁴ and R⁵, R⁵ and R⁶, or R⁶ and R⁷, taken together, may form a fused ring; R⁸ is a hydrocarbon group; and the symbol * is an asymmetric carbon atom), which comprises reacting an amine of formula (4): 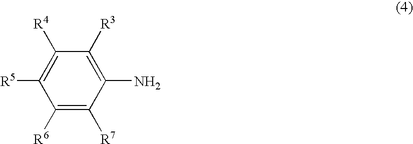
(wherein R³ is hydrogen, hydrocarbon, halogen, halogenated hydrocarbon, substituted hydrocarbon, aliphatic heterocyclic group, substituted aliphatic heterocyclic group, aromatic heterocyclic group, substituted aromatic heterocyclic group, alkoxy, substituted alkoxy, aralkyloxy, substituted aralkyloxy, aryloxy, substituted aryloxy, acyl, acyloxy, alkyloxycarbonyl, aryloxycarbonyl, aralkyloxycarbonyl, alkylenedioxy, nitro, amino, substituted amino, cyano, carboxyl, sulfo, sulfonyl or substituted silyl; R³ and R⁴, R⁴ and R⁵, R⁵ and R⁶, or R⁶ and R⁷, taken together, may form a fused ring, provided that either of R³ or R⁷ is hydrogen; and R⁴ to R⁷ are each the same as mentioned above) with an aldehyde of formula (5):
R²—CHO (5)
(wherein R² is the same as mentioned above), and reacting the resulting imine with a N-vinyl carbamate of formula (3): 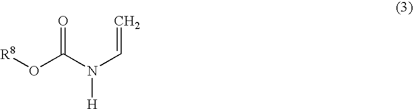
(wherein R⁸ is the same as mentioned above) in the presence of a chiral Lewis acid.

9. A mixture of an optically active amine of formula (9): 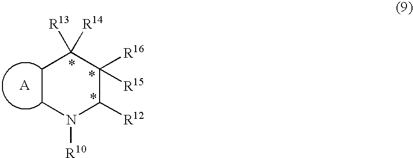
(wherein R¹⁰ is hydrogen, alkyl, aryl, substituted alkyl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹² is hydrocarbon, substituted hydrocarbon, COOR¹⁹ (R¹⁹ is a hydrocarbon group or a substituted hydrocarbon group), COR²⁰ (R²⁰ is a substituted amino group) or substituted amino; R¹³, R¹⁵ and R¹⁶ are each independently hydrogen or alkyl; R¹⁴ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, heteroaryloxy, substituted heteroaryloxy, alkylthio, substituted alkylthio, arylthio, substituted arylthio, aralkylthio, substituted aralkylthio, heteroarylthio, substituted heteroarylthio, substituted amino, substituted silyl, alkylseleno, aralkylseleno, arylseleno or heteroarylseleno; R¹⁴ and R¹⁶, taken together, may form a ring; the group represented by ring A is aryl, substituted aryl, aromatic heterocyclic group or substituted aromatic heterocyclic group; 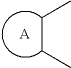
is a divalent group corresponding to the group represented by ring A as mentioned above; and the symbol * is an asymmetric carbon atom) and an optically active compound of formula (9b): 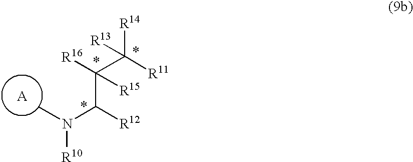
(wherein R¹¹ is a leaving group; the group represented by ring A, R¹⁰, R¹² to R¹⁶ and the symbol * are each the same as mentioned above).

10. A mixture of optically active amines of formula (10): 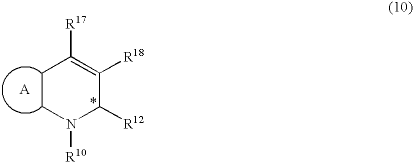
(wherein the group represented by ring A is aryl, substituted aryl, aromatic heterocyclic group or substituted aromatic heterocyclic group; R¹⁰ is hydrogen, alkyl, aryl, substituted alkyl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹² is hydrocarbon, substituted hydrocarbon, COOR¹⁹ (R¹⁹ is a hydrocarbon group or a substituted hydrocarbon group), COR²⁰ (R²⁰ is a substituted amino group) or substituted amino; R¹⁷ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, substituted amino or substituted silyl; R¹⁸ is hydrogen or alkyl; the symbol * is an asymmetric carbon atom; and 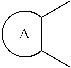
is a divalent group corresponding to the group represented by ring A as mentioned above) and an optically active compound (10b): 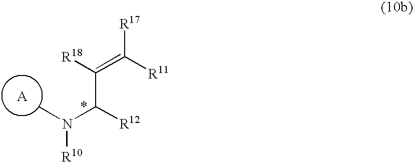
(wherein R¹¹ is a leaving group; R¹⁰, R¹², R¹⁷, R¹⁸ the symbol * and the group represented by ring A are each the same as mentioned above).

11. An optically active compound of formula (9c): 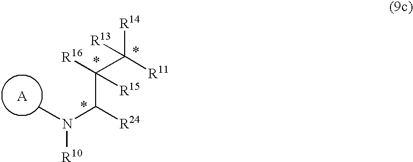
(wherein the group represented by ring A is aryl, substituted aryl, aromatic heterocyclic group or substituted aromatic heterocyclic group; R¹⁰ is hydrogen, alkyl, substituted alkyl, aryl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹¹ is a leaving group; R²⁴ is hydrocarbon, substituted hydrocarbon or COOR¹⁹ (R¹⁹ is a hydrocarbon group or a substituted hydrocarbon group), COR²⁰ (R²⁰ is a substituted amino group) or substituted amino; R¹³, R¹⁵ and R¹⁶ are each independently hydrogen or alkyl; R¹⁴ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, heteroaryloxy, substituted heteroaryloxy, alkylthio, substituted alkylthio, arylthio, substituted arylthio, aralkylthio, substituted aralkylthio, heteroarylthio, substituted heteroarylthio, substituted amino, substituted silyl, alkylseleno, aralkylseleno, arylseleno or heteroarylseleno; R¹⁴ and R¹⁶, taken together, may form a ring; the symbol * is an asymmetric carbon).

12. An optically active compound of formula (10c): 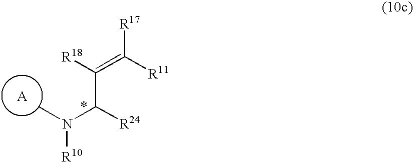
(wherein the group represented by ring A is aryl, substituted aryl, aromatic heterocyclic group or substituted aromatic heterocyclic group; R¹⁰ is hydrogen, alkyl, substituted alkyl, aryl, substituted aryl, acyl, substituted acyl, alkyloxycarbonyl, substituted alkyloxycarbonyl, aryloxycarbonyl, substituted aryloxycarbonyl, aralkyloxycarbonyl or substituted aralkyloxycarbonyl; R¹¹ is a leaving group; R²⁴ is hydrocarbon, substituted hydrocarbon or COOR¹⁹ (R¹⁹ is a hydrocarbon group or a substituted hydrocarbon group), COR²⁰ (R²⁰ is a substituted amino group) or substituted amino; R¹⁷ is aryl, substituted aryl, aliphatic heterocyclic group, substituted aliphatic heterocyclic group, aromatic heterocyclic group, substituted aromatic heterocyclic group, alkoxy, substituted alkoxy, aryloxy, substituted aryloxy, aralkyloxy, substituted aralkyloxy, substituted amino or substituted silyl; R¹⁸ is hydrogen or alkyl; the symbol * is an asymmetric carbon atom).

13. The optically active compound of formula (9c) according to claim 11, wherein the optically active compound of formula (9c) is an optically active compound of following formula: 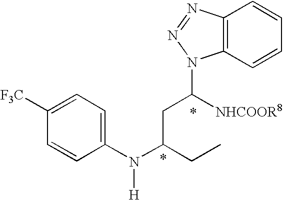
(wherein R⁸ and the symbol * are each the same as mentioned above).
